SEQUENCE LISTING (2 sequences)

SEQ ID NO: 1 - DNA, 31 bp, Artificial Sequence
  Synthetic aptamer
guggacuauaccgcguaaugcugccuccact

SEQ ID NO: 2 - DNA, 15 bp, Artificial Sequence
  Synthetic aptamer
cgcgguauaguccac

(19) United States
(12) Patent Application Publication
(10) Pub. No.: US 2009/0163437 A1
(43) Pub. Date: Jun. 25, 2009

(54) STEADY-STATE SUBCUTANEOUS ADMINISTRATION OF APTAMERS

(75) Inventor: Christopher P. Rusconi, Durham, NC (US)

Correspondence Address: KING & SPALDING, 1180 PEACHTREE STREET , NE, ATLANTA, GA, 30309-3521, US

(73) Assignee: REGADO BIOSCIENCES, INC.

(21) Appl. No.: 12/252,994

(22) Filed: Oct. 16, 2008

Related U.S. Application Data
(60) Provisional application No. 60/999,080, filed on Oct. 16, 2007.

(51) Int. Cl. A61K 31/7088

(52) U.S. Cl. 514/44

(57) ABSTRACT

An improved method of administration of an aptamer and modulator system to regulate blood coagulation in a host is provided wherein the aptamer is administered subcutaneously and the modulator is administered either subcutaneously or intravenously. This method for sustained aptamer activity using intermittent subcutaneous injections further allows for titrated modulation of the aptamer activity by administration of the modulator.

### CROSS-REFERENCE TO RELATED APPLICATIONS

This application, claims priority to U.S. Provisional Application No. 60/999,080, filed Oct. 16, 2007 and incorporated herein in its entirety.

### FIELD OF THE INVENTION

An improved method of administration of an aptamer and modulator system to regulate blood coagulation in a host is provided allowing for sustained anticoagulant or antithrombotic activity using intermittent subcutaneous injections.

### BACKGROUND

Given the central role of thrombosis in the pathobiology of acute ischemic heart disease, injectable anticoagulants have become the foundation of medical treatment for patients presenting with acute coronary syndromes (ACS), such as unstable angina and myocardial infarction, and for those undergoing coronary revascularization procedures (Harrington et ah, 2004; Popma et al., 2004).

In addition to acute ischemic conditions, venous thrombosis or pulmonary embolism affect approximately 500,000 people in the US each year. Venous thrombosis is a condition in which a blood clot forms in a vein, which can limit blood flow and cause swelling and pain. Most commonly, venous thrombosis occurs in the “deep veins” in the legs, thighs, or pelvis (deep vein thrombosis, or DVT). If a part or all of the blood clot in the vein breaks off from the site where it was created, it can travel through the venous system and cause an embolus. If the embolus lodges in the lung, it is called pulmonary embolism, a serious condition that leads to over 50,000 deaths a year in the United States. Symptoms of venous thrombosis can also include transient ischemic attacks or premature stroke. Patients with peripheral vascular disease, particularly lower extremity occlusive disease, are at higher risk; however, certain conditions including pregnancy also increase the risk of venous thrombosis. Venous thrombosis is a multifactorial condition caused by a combination of genetic, acquired or environmental influences. Excess clotting occurs when there is a disturbance in one of the coagulation inhibitor mechanisms or in natural lysis of clots. Known genetic causes explain about 50% of venous thrombosis cases and include a Factor V mutation (APC resistance) (approximately 20-40% of patients), Protein S deficiency (5-6% of patients), Protein C deficiency (2-5% of patients), Antithrombin III deficiency (2-4% of patients), Plasminogen deficiency (1-2% of patients), Heparin cofactor II deficiency (<1% of patients), or unknown genetic defect (approximately 40% of patients). Acquired or environmental conditions that can precipitate a thrombotic event include: pregnancy, oral contraceptive use, estrogen therapy, obesity, a malignancy, diabetes mellitus; venous stasis from immobility, trauma, a post-operative state, and Lupus anticoagulant.

Anticoagulants also play a central role in peripheral and cerberovascular diseases. Over 200,000 peripheral interventions were performed in 1997 and this number continues to grow at a rapid pace. Peripheral artery disease, also referred to as peripheral vascular disease, affects 12-20% of Americans over 65. Many of these people will be candidates for percutaneous peripheral intervention. Unfractionated heparin has been the antithrombotic of choice for percutaneous coronary intervention since the first reported angioplasty by Andreas Gruentzig in 1979. Despite significant therapeutic and technological advances, ischemic and hemorrhagic complications remain the most commonly associated risks during intervention and are a major source of morbidity, mortality and costs. Major and minor hemorrhagic complications range from 0.4-17%. (Aguirre and Gill (2002) J Invas Cardiol 14:48B-54B) and ischemic complications, measured by the combined clinical endpoint of death, myocardial infarction (MI) or revascularization, continue to be reported in from approximately 7.5% in the low-risk patients to over 14.0% in the high-risk, patients with heparin as anticoagulant. (The CAPTURE study. (1997) Lancet 349:1429-1435; Topol et al. (2001) N Engl J Med 344:1888-1894; Bittl et al. (2001) Am Heart J 142:952-959).

In addition to peripheral vascular disease, current therapies fail in anticoagulant needs for cerebrovascular disease. One of the major dilemmas in the field is the ideal timing to restart anticoagulant therapy in patients who have suffered an intracranial hemorrhage. Despite the lack of available data, most reports agree that 1) anticoagulant therapy has to be immediately reversed to decrease the risk of hemorrhage progression: 2) a period between 1 and 2 weeks appears sufficient to allow for management and monitoring of the hemorrhage off anticoagulant therapy; and 3) anticoagulant therapy can be safely restarted after the period off of treatment. A physician confronting a patient with intracranial hemorrhage and the need for anticoagulant therapy faces a situation of individually focused clinical decision making. The problem rests in balancing the risks of a worsening or recurring hemorrhage on one side, and the risk of thromboembolism on the other. A major need for this type of therapy is that anticoagulation should be immediately reversible and the reversal should be titratable.

Currently available anticoagulants include unfractionated heparin (UFH), the low molecular weight heparins (LMWH), and the direct thrombin inhibitors (DTI) such as recombinant hirudin, bivalirudin, and argatroban. The present paradigm both for anticoagulant use and for continued antithrombotic drug development is to establish a balance between efficacy, which means reducing the risk of ischemic events, and safety, which means minimizing the risk of bleeding (Harrington et al., 2004). Each of the available agents carries an increased risk of bleeding relative to placebo.

The major adverse event associated with anticoagulant and antithrombotic drugs is bleeding, which can cause permanent disability and death (Ebbesen et al., 2001; Levine et al., 2004). Generally, cardiovascular clinicians have been willing to trade off an increased risk of bleeding when a drug can reduce the ischemic complications of either the ACS or of coronary revascularization procedures. However, recent data have suggested that bleeding events, particularly those that require blood transfusion, have a significant impact on the outcome and cost of treatment of patients with ACS. Transfusion rates in patients undergoing elective coronary artery bypass graft (CABG) surgery range from 30-60%, and transfusion in these patients is associated with increased short, medium and long-term mortality (Bracey et al., 1999; Engoren et al., 2002; Hebert et al., 1999). Bleeding is also the most frequent and costly complication associated with percutaneous coronary-interventions (PCI), with transfusions being performed in 5-10% of patients at ah incremental cost of $8000-$12,000 (Moscucci, 2002). In addition, the frequency of significant bleeding in patients undergoing treatment for ACS is high as well, ranging from 5% to 10% (excluding patients who undergo CABG), with bleeding and transfusion independently associated with a significant increase in short-term mortality (Moscucci et al., 2003; Rao et al., 2004). Therefore, despite the continued development of novel antithrombotics, a significant clinical need exists for safer anticoagulant agents.

Rapid reversal of drug activity can be achieved passively by formulation of a drug as an infusible agent with a short half-life with termination of infusion as the means to reverse, or actively via administration of a second agent, a modulator, that can neutralize the activity of the drug. For hospitalized patients with acute ischemic heart disease, the ideal anticoagulant would be deliverable by intravenous or subcutaneous injection, immediately effective, easily dosed so as not to require frequent monitoring and immediately and predictably reversible.

UFH is the only reversible anticoagulant currently approved for use. However, UFH has significant limitations. First, heparin has complex pharmacokinetics that make the predictability of its use challenging (Granger et al., 1996). Second, the dose predictability of its modulator, protamine, is challenging, and there are serious side effects associated with its use (Carr and Silverman, 1999; Welsby et al., 2005). Finally, heparin can induce thrombocytopenia (HIT) and thrombocytopenia with thrombosis (HUT) (Warkentin, 2005; Warkentin and Greinacher, 2004).

Despite these limitations, heparin remains the most commonly used anticoagulant for hospitalized patients primarily because it is “reversible.” Newer-generation anticoagulants, such as the LMWHs have improved upon the predictability of UFH dosing and do not require lab-based monitoring as part of their routine use. HIT and HITT are observed less frequently with the LMWHs, relative to UFH, but they have not eliminated this risk. Two of the three commercially available DTIs, lepirudin and argatroban, are specifically approved for use in patients who have developed or have a history of HIT. Bivalirudin is approved for use as an anticoagulant during PCI and therefore provides an attractive alternative to UFH in patients who have HIT. However, there are no direct and clear ways to reverse the anticoagulant effects of the LMWHs, nor of the DTIs, which presents a particular risk to their use in patients undergoing surgical or percutaneous coronary revascularization procedures (Jones et al., 2002). Bleeding in patients treated with LMWH's or DTI's is managed by administering blood products, including clotting factors.

### Blood Coagulation and FIX

The cell-based model of coagulation (FIG. 1) provides the clearest explanation to date of how physiologic coagulation occurs in vivo (Hoffman et al., 1995; Kjalke et al., 1998; Monroe et al., 1996).

According to this model, the procoagulant reaction occurs in three distinct steps: initiation, amplification and propagation. Initiation of coagulation takes place on tissue factor-bearing cells such as activated monocytes, macrophages, and endothelial cells. Coagulation factor VIIa, which forms a complex with tissue factor, catalyzes the activation of coagulation factors IX (FIX) and X (FX), which in turn generates a small amount of thrombin from prothrombin. In the amplification phase (also referred to as the priming phase), the small amount of thrombin generated in the initiation phase activates coagulation factors V, VIII, and XI and also activates platelets, which supplies a surface upon which further procoagulant reactions occur. In vivo, the small amounts of thrombin generated during the amplification phase are not sufficient to convert fibrinogen to fibrin, due to the presence of endogenous thrombin inhibitors termed serpins, such as anti-thrombin III, α-2-macroglobulin and heparin cofactor II. The final phase of the procoagulant reaction, propagation, occurs exclusively on the surface of activated platelets. During propagation, significant amounts of FIXa are generated by the FXIa-catalyzed activation of FIX. FIXa forms a complex with its requisite cofactor FVIIIa, which activates FX. Subsequently, FXa forms a complex with its requisite cofactor FVa. The FXa-FVa complex activates prothrombin, which leads to a “burst” of thrombin generation and fibrin deposition. The end result is the formation of a stable clot.

Based upon this model, FIXa play two roles in coagulation. In the initiation phase, FIXa plays an important role in generating small amounts of thrombin via activation of FX to FXa and subsequent prothrombin activation. However, this role of FIXa is at least partially redundant with the tissue factor FVIIa-catalyzed conversion of FX to FXa. The more critical role of FIXa occurs in the propagation phase, in which the FVIIIa/FIXa enzyme complex serves as the sole catalyst of FXa generation on the activated platelet surface. Therefore, a reduction in FIXa activity, either due to genetic deficiency in FIX (i.e. hemophilia B) or pharmacologic inhibition of FIX/IXa, is expected to have several effects on coagulation. First, inhibition or loss of FIXa activity should partially dampen the initiation of coagulation. Second, inhibition or loss of FIXa activity should have a profound effect on the propagation phase of coagulation, resulting in a significant reduction or elimination of thrombin production. Finally, limitation of thrombin generation during the propagation phase will at least partially quell feedback amplification of coagulation by reducing activation of platelets and upstream coagulation factors such as factors V, VIII and XI.

### Prior Animal and Human Evaluation of Inhibitors of FIXa

Inhibitors of FIX activity, such as active site-inactivated factor IXa (FIXai) or monoclonal antibodies against FIX (e.g., the antibody BC2), have exhibited potent anticoagulant and antithrombotic activity in multiple animal models, including various animal models of arterial thrombosis and stroke (Benedict et al., 1991; Choudhri et al., 1999; Feuerstein et al., 1999; Spanier et al., 1998a; Spanier et al., 1997; Spanier et al., 1998b; Toomey et al., 2000). In general, these studies have shown that FIXa inhibitors have a higher ratio of antithrombotic activity to bleeding risk than unfractionated heparin in animals. However, in these studies, at doses marginally higher than the effective dose, animals treated with these agents have exhibited bleeding profiles no different than heparin. Such an experience in well-controlled animal studies suggests that, in the clinical setting, the ability to control the activity of a FIXa inhibitor would enhance its safety and facilitate its medical use. In addition, FIXai has been shown to be safe and effective as a heparin replacement in multiple animal surgical models requiring anticoagulant therapy, including rabbit models of synthetic patch vascular repair, as well as canine and non-human primate models of CABG with cardiopulmonary bypass (Spanier et al., 1998a; Spanier et al., 1997; Spanier et al., 1998b). FIXai has also been used successfully for several critically ill patients requiring cardiopulmonary bypass and in the setting of other extracorporeal circuits such as extracorporeal membrane oxygenation (Spanier et al., 1998a) by physicians at the Columbia College of Physicians and Surgeons, on a compassionate care basis. Thus, FIXa is a validated target for anticoagulant therapy in coronary revascularization procedures (both CABG and PCI), and for the treatment and prevention of thrombosis.

### Aptamer Development Drug-Modulator Pairs, and REG1

One approach to providing controlled, anticoagulation is the utilization of an anticoagulation agent with medium- to long-term duration of action of ˜12 hours and greater that can achieve clinically appropriate activity at relatively low doses, in combination with a second agent capable of specifically binding to and neutralizing the primary anticoagulant. Such a “drug-modulator” combination can ensure predictable and safe neutralization and reversal of the anticoagulant activity of the drug (Rusconi et al., 2004, Nat. Biotechnol. 22(11):1423-8; Rusconi et al., 2002, Nature 419(6902):90-4).

The drug-modulator technology has been applied to the discovery of aptamer based, anticoagulation systems such as REG1. Aptamers are single-stranded nucleic acids that bind with high affinity and specificity to target proteins (Nimjee et al., 2005), much like monoclonal antibodies. However, in order for an aptamer to bind to and inhibit a target protein, the aptamer must adopt a specific globular tertiary structure. Formation of this globular tertiary structure requires the aptamer to adopt the proper secondary structure (i.e., the correct base-paired and non-base-paired regions). As shown in cartoon form in FIG. 2, introduction of an oligonucleotide modulator complementary to a portion of an aptamer can change the aptamer's structure such that it can no longer bind to its target protein, and thus effectively reverses or neutralizes the pharmacologic activity, of the aptamer drug (Rusconi et al., 2004, Nat. Biotechnol. 22(11): 1423-8; Rusconi et al., 2002,Nature 419(6902):90-4). The REG1 system has been the subject of a phase I clinical trial to determine the safety profile and to characterize the pharmacodynamic responses using injections of the aptamer and modulator. Dyke, et al. (2006) Circulation 14:2490-7; Chan, et al. (2008) Circulation 117:2865-2874; Chan, et al. (2008) J. Thromb. Haemo, 6: 789-796 The drug component of REG1, RB006 (β-L-guggaCUaUaCCgCgUaaUgCuGcCUccacT wherein P= mPEG2-NHS ester MW 40 kDa; L= C6 NH₂ linker; G= 2-OH G; g= 2′-O—Me G; C= 2-F C; c= 2′-O-Me C; U= 2-F U: u= 2′-O—Me U; a= 2-O—Me A; and T= inverted 2′-H T (SEQ ED NO I); see FIG. 2), is a direct inhibitor of coagulation factor IXa (FIXa) that binds FIXa with high affinity and specificity (Rusconi et al., 2004, Nat Biotechnol. 22(11): 1423-8; Rusconi et al., 2002, Nature 419(6902):90-4; see also WO05/106042 to Duke University). RB006 elicits an anticoagulant effect by blocking the FVIIIa/FIXa-catalyzed conversion of FX to FXa. RB006 is a modified RNA aptamer, 31 nucleotides in length, which is moderately stabilized against endonuclease degradation by the presence of 2′-fluoro and 2′-O-methyl sugar-containing residues, and stabilized against exonuclease degradation by a 3′ inverted deoxythymidine cap. The nucleic acid portion of the aptamer is conjugated to a 40-kilodalton polyethylene glycol (PEG) carrier to enhance its blood half-life. Following bolus IV injection, the half-life of RB006 in mice is approximately 8 hours and in monkeys, approximately 12 hours. As such, RB006 can be given as a one-time bolus injection, rather than by IV infusion, to maintain an anticoagulated state over several hours.

As shown in FIG. 2, RB007 (cgcgguauaguccac wherein g= 2′-O—Me G; c= 2′-O-Me C; u= 2′-O—Me U; and a= 2′-O—Me A (SEQ ID NO 2); see FIG. 2), the modulator component of REG1, is an oligonucleotide complementary to a portion of RB006 that can effectively bind to RB006 and thereby neutralize its anti-FIXa activity. RB007 is a 2′-O-methyl RNA oligonucleotide 15 nucleotides in length that is complementary to a portion of the drug component of REG1. The 2′-O-methyl modification confers moderate nuclease resistance to the modulator, which provides sufficient in vivo stability to enable it to seek and bind RB006, but does not support extended in vivo persistence.

### Nonclinical Development of REG1

Pharmacology data demonstrating the specificity of the RB006 aptamer for FIXa and the affinity of the RB007 modulator for the aptamer has been developed. The results of these studies can be summarized as follows: the drug component of REG1 (RB006 and/or related precursor compounds) can: (1) effectively inhibit coagulation factor X activation in vitro; (2) prolong plasma clotting times in vitro in plasma from humans and other animal species; (3) systemically anticoagulate animals following bolus intravenous administration; (4) prevent thrombus formation in an animal arterial damage thrombosis model; (5) replace heparin in an animal cardiopulmonary bypass model; and (6) be effectively re-dosed in animals within 30 minutes following neutralization by the REG1 modulator component.

Nonclinical pharmacology studies to date have shown that RB007 can: (1) rapidly and durably neutralize the anticoagulant activity of the drug component of REG1 (RB006) in vitro in plasma from humans and other animal species; (2) rapidly and durably neutralize the anticoagulant activity of the drug component of REG1 in vivo following bolus IV administration in animals systemically anticoagulated with this agent; (3) prevent hemorrhage induced by a combination of supratherapeutic doses of the REG1 drug component and surgical trauma: and (4) neutralize the anticoagulant activity of the REG1 drug component in animals following cardiopulmonary bypass. Furthermore, the modulator has not exhibited any anticoagulant or other pharmacologic activity in vitro in human plasma, or in animals following bolus IV administration.

There remains a need to provide a reliable method of administration which allows for the predictable and repeatable effect of an aptamer-modulator system in a convenient and readily deliverable dosing regimen.

### SUMMARY OF THE INVENTION

It has been found that subcutaneous administration of an anti-coagulant aptamer, when compared to intravenous administration, results in a slower absorption phase and increased plasma residence times. Furthermore, it has been found that subsequent administration of a modulator following subcutaneous administration of the aptamer can temporarily reverse the anti-coagulant effect. These unexpected results allow for improved methods of administering anti-coagulant aptamers or aptamer/modulator systems as well as novel uses in the treatment of various thrombotic disorders. In one embodiment, a method of reduction or prevention of a thrombosis is provided comprising administering an aptamer to a host in a single subcutaneous dose less than once a day. In some embodiments, the aptamer is administered every two days. In other embodiments, the aptamer is administered every three days. In yet further embodiments, the aptamer is administered every four days, or once a week. In certain embodiments, the aptamer is administered for at least one week. In certain other embodiments, the aptamer is administered for at least two weeks, or at least one month. In some embodiments, the aptamer is administered at least six months. In some embodiments, the aptamer is administered in a depot that provides single doses without repeated subcutaneous injections.

In certain specific embodiments, the aptamer is pegylated. In specific embodiments, the aptamer is a pegylated RB006 (SEQ ED NO: 1). In one embodiment, the present invention provides an improved method of administration of an aptamer comprising: 1) determining a dose for a desired pharmacodynamic response; 2) administrating to the host a dose of aptamer anticoagulant to achieve the desired pharmacodynamic response, where the aptamer is administered in a single subcutaneous dose less than once a day. In certain instances the aptamer is as administered in a single subcutaneous dose every two days. In other instances the aptamer is administered in a single subcutaneous dose every three days or more.

In certain embodiments, a high level of anti-coagulation effect is desired. In those instances, an aptamer can be provided at a level of 2 to 20 mg/kg or greater. In other instances, a high intermediate level of anticoagulation effect is desired. In those instances, a dose of about between 1 to 15 mg/kg is provided to the host. In other instances, a low intermediate level of anti-coagulation is desired. In these instances, a dose of about 0.5 to 10 mg/kg is provided to the host. In other instances, a low level of anti-coagulation is desired. In these instances, a dose of about 0.05 to 1 mg/kg is provided to the host.

In certain embodiments, the anti-coagulation effect can be temporarily or permanently reversed by subsequent administration of an modulator to the aptamer. In certain instances the dose of modulator is provided based on a ratio with the dose of aptamer previously administered, adjusted for a desired reduction in aptamer activity. In other instances, this close of modulator is adjusted based on the time after administration of the aptamer. In certain instances, the ratio of modulator to aptamer is halved if the aptamer has been administered more than 24 hours previously.

In a principle embodiment, the aptamer anticoagulant system is the REG1 system, which comprises an aptamer anticoagulant and an oligonucleotide modulator. In certain, non-limiting embodiments, the aptamer is RB006 (SEQ ID NO: 1) and the modulator is RB007 (SEQ ID NO: 2). In one embodiment, the pharmacodynamic response is measured in coagulation assays such as the plasma or whole blood-based activated partial thromboplastin time (APTT) assay or the whole blood-based activated clotting time (ACT) assay, and can be reported as the absolute value, the percent effect, percent change, time weighted average or area under the curve over a defined time period.

In certain embodiments, the modulator is an antidote or reversal agent. The modulator can be an oligonucleotide, and in certain embodiments is anoligonucleotide that was identified utilizing SELEX or a similar process. In other embodiments, the modulator is a small molecule, peptide, polymer, oligosaccharide, lipid, nanoparticle or microsphere. In some embodiments, the modulator acts upon a specific aptamer anticoagulant and does not interact with other aptamers or nucleic acids or proteins in the host. In other embodiments, the modulator is non-specific and may act upon a variety of therapeutic aptamers.

The level of pharmacodynamic response can be at any level desired for a particular application. For example, in certain instances when a patient is at low risk for a thrombotic event, a low level of response may be desired. In particular instances, it may not be desirable to maximize clotting factor inhibition, and in particular FIX or FIXa inhibition by using a saturating amount of anticoagulant, particularly an aptamer to FIXa such as RB006. In other instances, when a patient is at a high risk for a thrombotic event or is having a thrombotic episode, a high level of response may be desired. In such instances, it may be desirable to maximize clotting factor inhibition, and in particular, FIX or FIXa inhibition, by using a saturating amount of anticoagulant, particularly an aptamer to FIXa such as RB006.

In one embodiment, an anticoagulant aptamer, such as RB006, is provided in daily or less than daily subcutaneous doses. In another embodiment, after subcutaneous delivery of the aptamer, a modulator is injected.

The procedures described herein allow for a step wise delivery of both anticoagulant and modulator to allow titration of either or both compounds to a desired level of target inhibition and reversal.

The ratio of modulator to aptamer is adjusted based on the desired level of inhibition of the aptamer. The modulator dose can be calculated based on correlation with the dose of aptamer. In one embodiment, the weight-to-weight ratio of modulator to aptamer is 1:1. In other embodiments, the ratio of modulator to aptamer is greater than 1:1 such as 2:1 or about 2:1, 3:1 or about 3:1, 4:1 or about 4:1, 5:1 or about 5:1, 6:1 or about 6:1, 7:1 or about 7:1, 8:1 or about 8:1, 9:1 or about 9:1, 10:1 or about 10:1 or more. These ratios can also be calculated based on modulator to aptamer ratio, which can, for example, be less than about 1:1 such as 0.9:1 or about 0.9:1, 0.8:1 or about 0.8:1, 0.7:1 or about 0.7:1, 0.6:1 or about 0.6:1, 0.5:1 or about 0.5:1, 0.45:1 or about 0.45:1, 0.4:1 or about 0.4:1, 0.35:1 or about 0.35:1, 0.3:1 or about 0.3:1, 0.25:1 or about 0.25:1, 0.2:1 or about 0.2:1, 0.15:1 or about 0.15:1, 0.1:1 or about 0.1:1 or less than 0.1:1 such as about 0.005:1 or less. In some embodiments, the ratio is between 0.5:1 and 0.1:1, or between 0.5:1 and 0.2:1, or between 0.5:1 and 0.3:1. In other embodiments, the ratio is between 1:1 and 5:1, or between 1:1 and 10:1, or between 1:1 and 20:1.

In some embodiments, only a partial reversal of aptamer activity occurs. For example, in some embodiments, aptamer activity is reversed by 90%, or less than 90% such as about 80%, about 70%, about 60%, about 50%, about 40%, about 30%, about 20%, about 10% or less. The ratio of modulator to aptamer can be calculated either by comparing weight to weight or on a molar basis. In certain embodiments, a self-administration kit is provided for outpatient use comprising a dosage unit of RB006; a needle for subcutaneous injection; and instructions for providing a steady state level of anticoagulation. The instructions will provide for less than daily subcutaneous-dosing. In certain embodiments, the kit will contain multiple, single use dosage units. In other embodiments, the dosage unit will comprise at least five separate administrations of RB006. In some embodiments, the kit will also comprise a dosage unit of modulator.

In particular embodiments of the invention, the host or subject to which the dosing system is applied is a human. In specific embodiments, the host is a human who is in need of anticoagulant therapy. In certain embodiments, the host is a human patient with one or more thrombotic disorders.

### BRIEF DESCRIPTION OF THE FIGURES

FIG. 1 depicts a cell based model of coagulation. TF—tissue factor; vWF—von Willebrands factor; II—prothrombin; IIA—thrombin; Va, VTIa, VIIIa, LXa, Xa, XIa—activated forms of coagulation factors V, VII, VIII, IX, X, and XI.

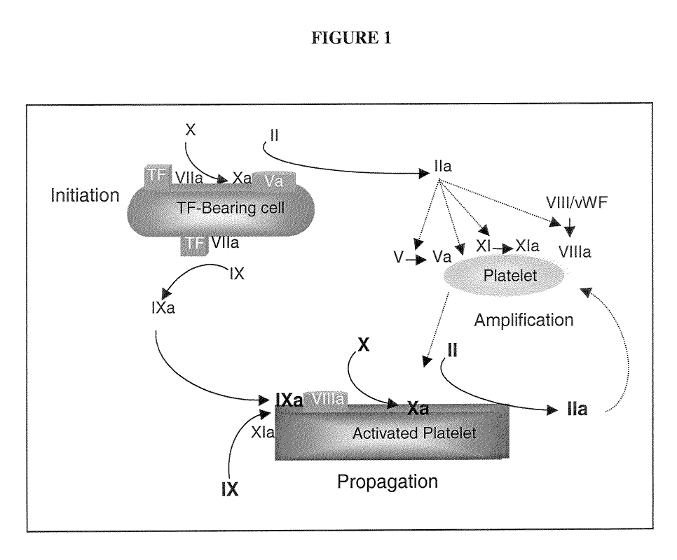

FIG. 2 depicts the REG1 anticoagulation system. The system is composed of the FIXa inhibitor RB006 and its matched modulator RB007. Recognition of the drug by the modulator is via Watson-Crick base pairing as shown. RB006 is a modified RNA aptamer composed of 2′-fluoro residues (upper case) 2′-O-methyl residues (lower case) and a single 2′-hydroxyl residue (underlined). RB006 is conjugated to a 40-KDa polyethylene glycol earner (P) via a 6-carbon amino acid linker (L), and is protected from exonuclease degradation by an inverted deoxythymidine on the 3′ end (idT). RB007 (the modulator or antidote) is a 2′-O-methyl-modified RNA oligonucleotide.

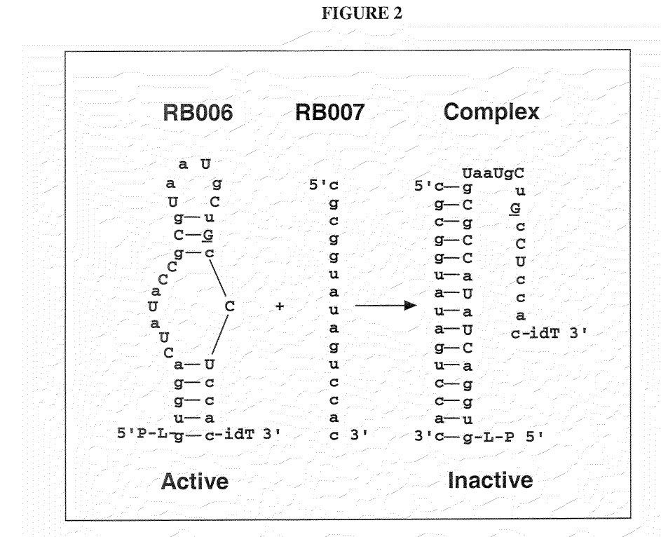

FIG. 3 is a graph of RB006 APTT dose response curve in vivo, showing the time course of absorption of RB006 following subcutaneous (SC) administration to Cynomolgus Monkeys. The concentration of RB006 in the plasma compartment was followed using the concentration-dependent APTT assay. Data are presented as the mean±SEM from two independent measurements.

FIG. 4 is a graph of RB006 APTT dose response curve in vivo, comparing the time course of absorption of RB006 following subcutaneous (SC) or intravenous (IV) injection in Cynomolgus Monkeys. The concentration of RB006 in the plasma compartment was followed using the concentration-dependent APTT assay. Data are presented as the mean±SEM from two independent measurements.

FIG. 5 is a graph showing the drug neutralizing activity of modulator RB007. RB007 (4 or 20 mpk) was injected intravenously 8 hours post subcutaneous injection of RB006 (2 or 10 mpk). Data are presented as the mean±SEM from two independent measurements. Anti-coagulant activity is measured using the APTT assay.

FIG. 6 is a graph of systemic anticoagulation of monkeys following administration of RB006 intravenously (15 or 30 mg/kg) and reversal with modulator RB007 (30 or 60 mg/kg). The level of anticoagulation in the monkeys was monitored with the APTT assay. RB007 was administered at t=3 hours following RB006 administration. Data are presented as the mean±SEM from two independent measurements.

FIG. 7 is graph of the time course of activity of repeated dosing of RB006 aptamer. The aptamer was administered subcutaneously every 48 hours. The level of anticoagulation activity in the monkeys is monitored with the APTT assay. Data are presented as the mean±SEM from two independent measurements.

### DETAILED DESCRIPTION

It has been found that subcutaneous administration of an anti-coagulant aptamer, when compared to intravenous administration, results in slower absorption and increased plasma residence times. Furthermore, it has been found that subsequent intravenous administration of a modulator following subcutaneous administration of the aptamer can temporarily reverse the anti-coagulant effect. These unexpected results allow for improved methods of administering anti-coagulant aptamers or aptamer/modulator systems as well as novel uses in the treatment of various thrombotic disorders.

### Development Of Aptamers

The term “aptamer” refers to a nucleic acid that binds to or otherwise interacts with a target protein. Aptamers can be therapeutic aptamers that bind to and inhibit a target protein useful in treatment or prophylaxis of a disease or condition, such as for inhibiting coagulation in a host in need thereof.

Nucleic acid aptamers are isolated using the Systematic Evolution of Ligands by Exponential Enrichment, termed SELEX, process. This method allows the in vitro evolution of nucleic acid molecules with highly specific binding to target molecules. The SELEX method is described in, for example, U.S. Pat. No. 7,087,735, U.S. Pat. No. 5,475,096 and U.S. Pat. No. 5,270,163, (see also WO 91/19813).

The SELEX method involves selection from a mixture of candidate oligonucleotides and step-wise iterations of binding, partitioning and amplification, using the same general selection scheme, to achieve virtually any desired criterion of binding affinity and selectivity. Starting from a mixture of nucleic acids, such as mixtures comprising a segment of randomized sequence, the SELEX method includes steps of contacting the mixture with the target under conditions favorable for binding, partitioning unbound nucleic acids from those nucleic acids which have bound specifically to target molecules, dissociating the nucleic acid-target complexes, amplifying the nucleic acids dissociated from the nucleic acid-target complexes to yield a ligand-enriched mixture of nucleic acids, then reiterating the steps of binding, partitioning, dissociating and amplifying through as many cycles as desired to yield highly specific, high affinity aptamers to the target molecule.

The basic SELEX method has been modified to achieve a number of specific objectives. For example, U.S. Pat. No. 5,707,796 describes the use of SELEX in conjunction with gel electrophoresis to select nucleic acid molecules with specific structural characteristics, such as bent DNA. U.S. Pat. No. 5,763,177 describes a SELEX-based method for selecting aptamers containing photoreactive groups capable of binding and/or photocrosslinking to and/or photoinactivating a target molecule. U.S. Pat. No. 5,580,737 describes a method for identifying highly specific aptamers able to discriminate between closely related molecules, termed Counter-SELEX. U.S. Pat. Nos. 5,567,588 and 5,861,254 describe SELEX-based methods which achieve highly efficient partitioning between oligonucleotides having high and low affinity for a target molecule. U.S. Pat. No. 5,496,938, describes methods for obtaining improved aptamers after the SELEX process has been performed. U.S. Pat. No. 5,705,337, describes methods for covalently linking a ligand to its target.

The feasibility of identifying aptamers to small peptides in solution was demonstrated in U.S. Pat. No. 5,648,214. The ability to use affinity elution with a ligand to produce aptamers that are targeted to a specific site on the target molecule is exemplified in U.S. Pat. No. 5,780,228, which relates to the production of high affinity aptamers binding to certain lectins. Methods of preparing aptamers to certain tissues, which include groups of cell types, are described in U.S. Pat. No. 6,127,119. The production of certain modified high affinity ligands to calf intestinal phosphatase is described in U.S. Pat. No. 6,673,553. U.S. Pat. No. 6,716,580 describes an automated process of identifying aptamers that includes the use of a robotic manipulators.

In its most basic form, the SELEX process may be defined by the following series of steps:

1) A candidate mixture of nucleic acids of differing sequence is prepared. The candidate mixture generally includes regions of fixed sequences (i.e., each of the members of the candidate mixture contains the same sequences in the same location) and regions of randomized sequences. The fixed sequence regions are selected either: (a) to assist in the amplification steps described below, (b) to mimic a sequence; known to bind to the target, or (c) to enhance the concentration of a given structural arrangement of the nucleic acids in the candidate mixture. The randomized sequences can be totally randomized (i.e., the probability of finding a base at any position being one in four) or only partially randomized (e.g., the probability of finding a base at any location can be selected at any level between 0 and 100 percent).

2) The candidate mixture is contacted with the selected target under conditions favorable for binding between the target and members of the candidate mixture. Under these circumstances, the interaction between the target and the nucleic acids of the candidate mixture can be considered as forming nucleic acid-target pairs between the target and those nucleic acids having the strongest affinity for the target.

3) The nucleic acids with the highest affinity for the target are partitioned from those nucleic acids with lesser affinity to the target. Because only an extremely small number of sequences (and possibly only one molecule of nucleic acid) corresponding to the highest affinity nucleic acids exist in the candidate mixture, it is generally desirable to set the partitioning criteria so that a significant amount of the nucleic acids in the candidate mixture (approximately 5 to 50%) are retained during partitioning.

4) Those nucleic acids selected during partitioning as having the relatively higher affinity to the target are then amplified to create a new candidate mixture that is enriched in nucleic acids having a relatively higher affinity for the target.

5) By repeating the partitioning and amplifying steps above, the newly formed candidate mixture contains fewer and fewer weakly binding; sequences, and the average degree of affinity of the nucleic acids to the target will generally increase. Taken to its extreme, the SELEX process will yield a candidate mixture containing one or a small number of unique nucleic acids representing those nucleic acids from the original candidate mixture having the highest affinity to the target molecule.

### Chemical Modifications

One problem encountered in the therapeutic, use of nucleic acids is that oligonucleotides in their phosphodiester form may be quickly degraded in body fluids by intracellular and extracellular enzymes such as endonucleases and exonucleases before the desired effect is manifest. Certain chemical modifications of the oligonucleotides, including aptamers, can be made to increase the in vivo stability of oligonucleotides or to enhance or to mediate the delivery of oligonucleotides. For the purposes of this section, the term oligonucleotide shall include, but not be limited to, aptamers as well as oligonucleotide modulators.

Modifications of the oligonucleotides include, but are not limited to, those which provide other chemical groups that incorporate additional charge, polarizability, hydrophobicity, hydrogen bonding, electrostatic interaction, and fluxionality to the aptamer bases or to the aptamer as a whole. Such modifications include, but are not limited to, 2′-position sugar modifications. 5-position pyrimidine modifications, 8-position purine modifications, modifications at exocyclic amines, substitution of 4-thiouridine, substitution of 5-bromo or 5-iodo-uracil; backbone modifications, phosphorothioate or alkyl phosphate modifications, methylations, unusual base-pairing combinations such as the isobases isocytidine and isoguanidine and the like. Modifications, can also include 3′ and 5′ modifications such as capping.

The SELEX method encompasses the identification of high-affinity aptamers containing modified nucleotides conferring improved characteristics on the ligand, such as improved in vivo stability or improved delivery characteristics. Examples of such modifications include chemical substitutions at the ribose and/or phosphate and/or base positions. SELEX-identified aptamers containing modified nucleotides are described in U.S. Pat. No. 5,660,985 that describes oligonucleotides containing nucleotide derivatives chemically modified at the 5- and 2′-positions of pyrimidines. U.S. Pat. No. 5,580,737 describes specific aptamers containing one or more nucleotides modified with 2′-amino (2′-NH2), 2′-fluoro (2′-F), and/or 2′-O-methyl (2′-OMe). U.S. Pat. No. 5,756,703, describes oligonucleotides containing various 2′-modified pyrimidines.

The SELEX method encompasses combining selected oligonucleotides with other selected oligonucleotides and non-oligonucleotide functional units as described in U.S. Pat. Nos. 5,637,459 and 5,683,867. U.S. Pat. No. 5,637,459 describes highly specific aptamers containing one or more nucleotides modified with 2′-amino. (2′—NH 2), 2′-fluoro (2′-F), and/or 2′-O-methyl (2′-OMe). The SELEX method further encompasses combining selected aptamers with lipophilic or Non-Immunogenic, High Molecular Weight compounds in a diagnostic or therapeutic complex as described in U.S. Pat. No. 6,011,020.

Where the aptamers are derived by the SELEX method, the modifications can be pre- or post-SELEX modifications. Pre-SELEX modifications can yield aptamers with both specificity for its target and improved in vivo stability. Post-SELEX modifications made to 2′-OH aptamers can result in improved in vivo stability without adversely affecting the binding capacity of the aptamers. In one embodiment, the modifications of the aptamer include a 3′-3′ inverted phosphodiester linkage at the 3′ end of the molecule and 2′ fluoro (2′-F) and/or 2′ amino (2′-NH2), and/or 2′ O methyl (2′-OMe) modification of some or all of the nucleotides.

In one embodiment, the aptamer or its modulator can be covalently attached to a lipophilic compound such as cholesterol, dialkyl glycerol, diacyl glycerol, or a non-immunogenic, high molecular weight compound or polymer such as polyethylene glycol (PEG). In these cases, the pharmacokinetic properties of the aptamer or modulator can be enhanced. In still other embodiments, the aptamer or the modulator can be encapsulated inside a liposome. The lipophilic compound or non-immunogenic; high molecular weight compound can be covalently bonded or associated through non-covalent interactions with aptamer or modulators). In embodiments where covalent attachment is employed, the lipophilic compound or non-immunogenic, high molecular weight compound may be covalently bound to a variety of positions on the aptamer or modulator, such as to an exocyclic amino group on the base, the 5-position of a pyrimidine nucleotide, the 8-position of a purine nucleotide, the hydroxyl group of the phosphate, or a hydroxyl group or other group at the 5′ or 3′ terminus. In one embodiment, the covalent attachment is to the 5′ or 3′ hydroxyl group. Attachment of the oligonucleotide modulator to other components of the complex can be done directly or with the utilization of linkers or spacers.

Oligonucleotides of the invention can be modified at the base moiety, sugar moiety, or phosphate backbone, for example, to improve stability of the molecule, hybridization, etc. The oligonucleotide can include other appended groups. To this end, the oligonucleotide can be conjugated to another molecule, e.g., a peptide, hybridization triggered cross-linking agent, transport agent, hybridization-triggered cleavage agent, etc. The oligonucleotide can comprise at least one modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2α-thiouracil, β-D-mannosylqueosine, 5′-methoxycarboxymethyluracil, 5-methoxyuracil, 2-methylthio-N&isopentenyladeninc, uracil oxyacetic acid, wybutoxosine, pseudouracil, queosine, 2-thiocytosine, 5-methyl thiouracil, 2-thiouracil, 4-thiouracil, 5-methyluracil, -uracil-5-oxyacetic acid methylester, uracil oxyacetic acid (v), 5-methyl thiouracil, 3-(3-amino-3-N carboxypropyl) and 2,6-diaminopurine.

The aptamer or modulator of the invention can also comprise at least one modified sugar moiety selected from the group including, but not limited to, arabinose, 2-fluoroarabinose, xylose, and hexose. The aptamer or modulator can comprise at least one modified phosphate backbone selected from the group including, but not limited to, a phosphorothioate, a phosphorodithioate, a phosphoramidothioate, a phosphoramidate, a phosphorodiamidate, a methylphosphonate, an alkyl phosphotriester, and a formacetal or analog thereof.

Any of the oligonucleotides of the invention can be synthesized by standard methods known in the art, e.g. by use of an automated DNA synthesizer (such as are commercially available from, for example, Biosearch, Applied Biosystems).

### Modulators

The modulators of the invention can be oligonucleotides, small molecules, peptides, polymers, oligosaccharides, for example aminoglycosides, or other molecules that can bind to or otherwise modulate the activity of the aptamer, or a chimera or fusion or linked product of any of these. In certain embodiments, the modulators are antagonists, i.e. they reduce or eliminate the physiological activity of the aptamer. Antagonists can also be referred to as ‘reversal agents’. In certain non-limiting embodiments, the modulator is an oligonucleotide.

In one embodiment, the modulator is an oligonucleotide complementary to at least a portion of the aptamer. In another embodiment, the modulator can be a ribozyme or DNAzyme that targets the aptamer. In a further embodiment, the modulator can be a peptide nucleic acid (PNA), morpholino nucleic acid (MNA), locked nucleic acid (LNA) or pseudocyclic oligonucleobases (PCO) that includes a sequence that is complementary to or hybridizes with at least a portion of the aptamer.

An aptamer possesses an active tertiary structure which is dependent on formation of the appropriate stable secondary structure. Therefore, while the mechanism of formation of a duplex between a complementary oligonucleotide modulator of the invention and an aptamer is the same as between two short linear oligoribonucleotides, both the rules for designing such interactions and the kinetics of formation of such a product are impacted by the intramolecular aptamer structure. The rate of nucleation is important for formation of the final stable duplex, and the rate of this step is greatly enhanced by targeting the oligonucleotide modulator to single-stranded loops and/or single-stranded 3′ or 5′ tails present in the aptamer. For the formation of the intermolecular duplex to occur, the free energy of formation of the intermolecular duplex has to be favorable with respect to formation of the existing intramolecular duplexes within the targeted aptamer.

Modulators can be designed so as to bind a particular aptamer with a high degree of specificity and a desired degree of affinity. Modulators can be also be designed so that, upon binding, the structure of the aptamer is modified to either a more or less active form. For example, the modulator can be designed so that upon binding to the targeted aptamer, the three-dimensional structure of that aptamer is altered such that the aptamer can no longer bind to its target molecule or binds to its target molecule with less affinity.

Alternatively, the modulator can be designed so that, upon binding, the three dimensional structure of the aptamer is altered so that the affinity of the aptamer for its target molecule is enhanced. That is, the modulator can be designed so that, upon binding, a structural motif is produced in the aptamer so that the aptamer can bind to its target molecule.

In an alternative embodiment of the invention, the modulator itself is an aptamer. In this embodiment, an aptamer is first generated that binds to the desired therapeutic target. In a second step, a second modulator aptamer that binds to the first aptamer is generated using the SELEX process described herein or other process, and modulates the interaction between the therapeutic aptamer and the target. In one embodiment, the second (modulator) aptamer deactivates the effect of the first (therapeutic) aptamer.

In other alternative embodiments; the aptamer which binds to the target can be a PNA, MNA, LNA or PCO and the modulator is an aptamer. Alternatively, the aptamer which binds to the target is a PNA, MNA, LNA or PCO, and the modulator is a PNA. Alternatively, the aptamer which binds to the target is a PNA, MNA, LNA or PCO, and the modulator is an MNA. Alternatively, the aptamer which binds to the target is a PNA, MNA, LNA or PCO, and the modulator is an LNA. Alternatively, the aptamer which binds to the target is a PNA, MNA, LNA or PCO, and the modulator is a PCO. Any of these can be used, as desired, in the naturally occurring stereochemistry or in non-naturally occurring stereochemistry or a mixture thereof. For example, in a preferred embodiment, the aptamer is in the D configuration, and in an alternative embodiment, the aptamer is in the L configuration.

Modulators can include pharmaceutically acceptable positively charged compounds, including proteins, lipids, and natural synthetic polymers that can bind an aptamer in, for example, biological fluids. Protamines are included in this group as they are proteins that yield basic amino acids on hydrolysis and that occur combined with nucleic acid in the sperm of fish, such as salmon. Protamines are soluble in water, are not coagulated by heat, and comprise arginine, alanine and serine (most also contain proline and valine and many contain glycine and isoleucine). Modulators also include protamine variants (see e.g., Wakefield et al, J. Surg. Res. 63:280 (1996)) and modified forms of protamine, including those described in U.S. Publication No. 20040121443. Other modulators include protamine fragments, such as those described in U.S. Pat. No. 6,624,141 and U.S. Publication No. 20050101532. Modulators also include, generally, peptides that modulate the activity of heparin, other glycosaminoglycans or proteoglycans (see, for example. U.S. Pat. No. 5,919,761). Examples of modulators are compounds that contain cationic-NH groups permitting stabilizing charge-charge interactions with a phosphodiester backbone. Certain exemplary modulators are poly-L-lysine, poly-L-ornithine, polyphosphoramidate or polyphosphoramidate containing polymers, polyethyleneimine, ionene, putrescine, spermine, poly(allylamine), dendrimers, polyamidoamine PAMAM, polypropyleneimine containing dendrimers or polymers such as a PPI dendrimer and cyclodextrin containing polymers. In certain embodiments, the modulators are compounds' as described in PCT Publication No. WO2008/121354.

In one embodiment, the modulator of the invention is an oligonucleotide that comprises a sequence complementary to at least a portion of the targeted aptamer sequence. For example, the oligonucleotide modulator can comprise a sequence complementary to 6-25 nucleotides of the targeted aptamer, typically, 8-20 nucleotides, more typically, 10-15 nucleotides. Advantageously, the oligonucleotide modulator is complementary to 6-25 consecutive nucleotides of the aptamer, or 8-20 or 10-15 consecutive nucleotides. The length of the oligonucleotide modulator can be optimized taking into account the targeted aptamer and the effect sought. Typically the oligonucleotide modulator is 5-80 nucleotides in length, more typically, 10-30 and most typically 15-20 nucleotides (e.g., 15-17). The oligonucleotide can be made with nucleotides bearing D or L stereochemistry, or a mixture thereof. Naturally occurring nucleosides are in the D configuration.

Various strategies can be used to determine the optimal site for oligonucleotide modulator binding to a targeted aptamer. An empirical strategy can be used in which complimentary oligonucleotides are “walked” around the aptamer. A walking experiment can involve two experiments performed sequentially; A new candidate mixture can be produced in which each of the members of the candidate mixture has a fixed nucleic acid-region that corresponds to a oligonucleotide modulator of interest. Each member of the candidate mixture also contains a randomized region of sequences. According to this method it is possible to identify what are referred to as “extended” oligonucleotides, which contain regions that can bind to more than one binding domain of an aptamer. In accordance with this approach, oligonucleotides (e.g., 2′-O-methyl oligonucleotides) about 15 nucleotides in length can be used that are staggered by about 5 nucleotides on the aptamer (e.g., oligonucleotides complementary to nucleotides 1-15, 6-20, 11-25, etc. of the aptamer). An empirical strategy can be particularly effective because the impact of the tertiary structure of the aptamer on the efficiency of hybridization of the oligonucleotide modulator can be difficult to predict. Assays described in the Examples that follow can be used to assess the ability of the different oligonucleotides to hybridize to a specific aptamer, with particular emphasis on the molar excess of the oligonucleotide modulator required to achieve complete binding to the aptamer. The ability of the different oligonucleotide modulators to increase the rate of dissociation of the aptamer from, or association of the aptamer with, its target molecule can also be determined by conducting standard kinetic studies using, for example, BIACORE assays. Oligonucleotide modulators can be selected such that a 5-50 fold molar excess of oligonucleotide, or less, is required to modify the interaction between the aptamer and its target molecule in the desired manner.

Alternatively, the targeted aptamer can be modified so as to include a single-stranded tail (3′ or 5′) in order to promote association with an oligonucleotide modulator. Suitable tails can comprise 1 to 20 nucleotides, preferably, 1-10 nucleotides, more preferably, 1-5 nucleotides and, most preferably, 3-5 nucleotides (e.g., 2′-O-methyl modified nucleotides). Tailed aptamers can be tested in binding and bioassays (e.g., as described in the Examples that follow) to verify that addition of the single-stranded tail does not disrupt the active structure of the aptamer. A series of oligonucleotides (for example, 2′-O-methyl oligonucleotides) that can form, for example, 1, 3 or 5 base pairs with the tail sequence can be designed and tested for their ability to associate with the tailed aptamer alone, as well as their ability to increase the rate of dissociation of the aptamer from, or association of the aptamer with, its target molecule. Scrambled sequence controls can be employed to verify that the effects are due to duplex formation and not non-specific effects.

The oligonucleotide modulators of the invention comprise a sequence complementary to at least a portion of an aptamer. However, absolute complementarity is not required. A sequence “complementary to at least a portion of an aptamer,” referred to herein, means a sequence having sufficient complementarity to be able to hybridize with the aptamer. The ability to hybridize can depend on both the degree of complementarity and the length of the hybridizing antisense oligonucleotide modulator. Generally, the larger the hybridizing oligonucleotide modulator, the more base mismatches with a target aptamer it can contain and still form a stable duplex (or triplex as the case may, be). One skilled in the art can ascertain a tolerable degree of mismatch by use of standard procedures to determine the melting point of the hybridized complex. In specific aspects, the oligonucleotide modulator can be at least 5 or at least 10 nucleotides, at least 15 or 17 nucleotides, at least 25 nucleotides or at least 50 nucleotides. The oligonucleotide modulators of the invention can be DNA or RNA or chimeric mixtures or derivatives or modified versions thereof, and can be single-stranded or double stranded.

In one embodiment, the modulator is a ribozyme or a DNAzyme. There are at least five classes of ribozymes that each display a different type of specificity. For example, Group I Introns are about 300 to >1000 nucleotides in size and require a U in the target sequence immediately 5′ of the cleavage site and binds 4-6 nucleotides at the 5′-side of the cleavage site. Another class are RNaseP RNA (M1 RNA), which are about 290 to 400 nucleotides in size. A third example are Hammerhead Ribozyme, which are about 30 to 40 nucleotides in size. They require the target sequence UH immediately 5′ of the cleavage site and bind a variable number nucleotides on both sides of the cleavage site. A fourth class are the Hairpin Ribozymes, which are about 50 nucleotides in size. They requires the target sequence GUC immediately 3′ of the cleavage site and bind 4 nucleotides at the 5-side of the cleavage site and a variable number to the 3′-side of the cleavage site. The fifth group are Hepatitis Delta Virus (HDV) Ribozymes, which are about 60 nucleotides in size.

Another class of catalytic molecules are called “DNAzymes”. DNAzymes are single-stranded, and cleave both RNA and DNA. A general model for the DNAzyme has been proposed, and is known as the “10-23” model. DNAzymes following the “10-23” model have a catalytic domain of 15 deoxyribonucleotides, flanked by two substrate-recognition domains of seven to nine deoxyribonucleotides each.

Nucleobases of the oligonucleotide modulators of the invention can be connected via internucleobase linkages, e.g., peptidyl linkages (as in the case of peptide nucleic acids (PNAs); Nielsen et al. (1991) Science 254, 1497 and U.S. Pat. No. 5,539,082) and morpholino linkages (Qin et al., Antisense Nucleic Acid Drug Dev. 10, 11 (2000); Summerton, Antisense Nucleic Acid Drug Dev. 7, 187 (1997); Summerton et al., Antisense Nucleic Acid Drug Dev. 7, 63 (1997); Taylor et al., J Biol. Chem. 271, 17445 (1996); Partridge et al., Antisense Nucleic Acid Drug Dev. 6, 169 (1996)), or by any other natural or modified linkage. The oligonucleobases can also be Locked Nucleic Acids (LNAs). Nielsen et al., J Biomol Struct Dyn 17, 175 (1999); Petersen et al., J Mol Recognit 13, 44 (2000); Nielsen et al., Bioconjug Chem 11, 228 (2000).

PNAs are compounds that are analogous to oligonucleotides, but differ in composition. In PNAs, the deoxyribose backbone of oligonucleotide is replaced with a peptide backbone. Each subunit of the peptide backbone is attached to a naturally-occurring or non-naturally-occurring nucleobase. PNA often has an achiral polyamide backbone consisting of N-(2-aminoethyl)glycine units. The purine or pyrimidine bases are linked to each unit via a methylene carbonyl linker (1-3) to target the complementary nucleic acid. PNA binds to complementary RNA or DNA in a parallel or antiparallel orientation following the Watson-Crick base-pairing rules. The uncharged, nature of the PNA oligomers enhances the stability of the hybrid PNA/DNA(RNA) duplexes as compared to the natural homoduplexes.

Morpholino nucleic acids are so named because they are assembled from morpholino subunits, each of which contains one of the four genetic bases (adenine, cytosine, guanine, and thymine) linked to a 6-membered morpholine ring. Eighteen to twenty-five subunits of these four subunit types are joined in a specific order by non-ionic phosphorodiamidate intersubunit linkages to give a morpholino oligo. These morpholino oligos, with their 6-membered morpholine backbone moieties joined by non-ionic linkages, afford substantially better antisense properties than do RNA, DNA, and their analogs having 5-membered ribose or deoxyribose backbone moieties joined by ionic linkages (see wwwgene-tools.com/Moiphol-inos/body_morpholinos.HTML).

LNA is a class of DNA analogues that possess some features that make it a prime candidate for modulators of the invention. The LNA monomers are bi-cyclic compounds structurally similar to RNA-monomers. LNA share most of the chemical properties of DNA and RNA, it is water-soluble, can be separated by gel electrophoreses, ethanol precipitated etc (Tetrahedron, 54, 3607-3630 (1998)). However, introduction of LNA monomers into either DNA or RNA oligos results in high thermal stability of duplexes with complementary DNA or RNA, while, at the same time obeying the Watson-Crick base-pairing rules. This high thermal stability of the duplexes formed with LNA oligomers together with the Finding that primers containing 3′ located LNA(s) are substrates for enzymatic extensions (e.g. the PCR reaction) is used in the present invention to significantly increase the specificity of detection of variant nucleic acids in the in vitro assays described in the application. The amplification processes of individual alleles occur highly discriminative (cross reactions are not visible) and several reactions may take place in the same vessel. See for example U.S. Pat. No. 6,316,198.

Pseudo-cyclic oligonucleobases (PCOs) can also be used as a modulator in the present invention (see U.S. Pat. No. 6,383,752), PCOs contain two oligonucleotide segments attached through their 3′-3′ or 5′-5′ ends. One of the segments (the “functional segment”) of the PCO has some functionality (e.g., an antisense oligonucleotide, complementary to a target mRNA). Another segment (the “protective segment”) is complementary to the 3′- or 5′-terminal end of the functional segment (depending on the end through which it is attached to the functional segment). As a result of complementarity between the functional and protective segment segments, PCOs form intramolecular pseudo-cyclic structures in the absence of the target nucleic acids (e.g., RNA). PCOs are more stable than conventional antisense oligonucleotides because of the presence of 3′-3′ or 5′-5′ linkages and the formation of intramolecular pseudo-cyclic structures. Pharmacokinetic; tissue distribution, and stability studies in mice suggest that PCOs have higher in vivo stability than and, pharmacokinetic and tissue distribution profiles similar to, those of PS-oligonucleotides in general, but rapid elimination from selected tissues. When fluorophore and quencher molecules are appropriately linked to the PCOs of the present invention, the molecule will fluoresce when it is in the linear configuration, but the fluorescence is quenched in the cyclic conformation.

Peptide-based modulators of aptamers represent an alternative molecular class of modulators to oligonucleotides or their analogues. This class of modulators may prove to be particularly useful when sufficiently active oligonucleotide modulators of a target aptamer cannot be isolated due to the lack of sufficient single-stranded regions to promote nucleation between the target and the oligonucleotide modulator. In addition, peptide modulators provide different bioavailabilities and pharmacokinetics than oligonucleotide modulators.

Oligosaccharides, like aminoglycosides, can bind to nucleic acids and can be used to modulate the activity of aptamers. A small molecule that intercalates between the aptamer and the target or otherwise disrupts or modifies the binding between the aptamer and target can also be used as the therapeutic regulator. Such small molecules can be identified by screening candidates in an assay that measures binding changes between the aptamer and the target with and without the small molecule, or by using an in vivo or in vitro assay that measures the difference in biological effect of the aptamer for the target with and without the small molecule. Once a small molecule is identified that exhibits the desired effect, techniques such as combinatorial approaches can be used to optimize the chemical structure for the desired regulatory effect.

In addition certain polymeric agents that bind nucleic acids can be used to modulate the activity of aptamers. Certain agents, which have been identified as potentially useful for providing controlled delivery of plasmid DNA's in the form of micro and nano spheres, may also be suitable modulators of short, structured RNA-based aptamers. The binding properties of these polymers have heretofore remained unrecognized as applicable to the in vivo modulation of aptamer pharmacology. In certain, embodiments, the modulator is a polymer, polysaccharide, lipid, nanoparticle or microsphere modulator.

The polymer, lipid, nanoparticle or microsphere modulator is generally an agent that has been known and used to complex with and deliver DNA plasmids and other nucleic acids in vivo. A variety of compounds are known in the art as potentially useful for delivery of DNA plasmids into a host. For delivery of DNA vaccines, nanoparticles are made in vitro and delivered by a variety of means (IV, intraperitoneal, intramuscular, subcutaneous, nasal, oral, etc.), with the DNA plasmid encapsulated in the nanoparticle. In specific embodiments, the modulator is a lipid. In other embodiments, the modulator is a polymer. In certain embodiments, the modulator is in the form of a nanoparticle. In other embodiments, the modulator is in the form of a microsphere.

In non-limiting examples, the modulator can be selected from the group consisting of: 1,2-dioleoyl-sn-glycero-3-ethylphosphocholine (EDOPC); dilauroylethylphosphatidylcholine (EDLPC); EDLPC/EDOPC; pyridinium surfactants; dioleoylphosphatidyl-ethanolamine (DOPE); (±)-N-(3-aminopropyl)-N,N-dimethyl-2,3-bis(dodecyloxy)-1-propanaminium bromide (GAP-DLRIE) plus the neutral co-lipid dioleoylphosphatidylethanolamine (DOPE) (GAP-DLRIE/DOPE); (±)-N,N-dimethyl-N-[2-(spermine carboxamido)ethyl]-2,3-bis(dioeyloxy-1-propaniminium petahydrochloride (DOSPA); dilauroylethylphosphatidylcholine (EDLPC); Ethyldimyristoyl phosphatidylcholine (EDMPC); (±)-N,N,N-trimethyl-2,3-bis(z-octadec-9-ene-oyloxy)-1-propanaminium chloride (DOTAP); (±)-N-2-(2-hydroxyethyl)-N,N-dimethyl-2,3-bis(tetradecyloxy)-1-propanaminium bromide (DMRIE); (±)-N,N,N-trimethyl-2,3-bis(z-octadec-9-enyloxy)-1-propanaminium chloride (DOTMA); 5-carboxyspermylglycine dioctadecyl-amide (DOGS); dipalmitoylphosphatidylethanolamine 5-carboxyspermylamide (DPPES); 1,3 dioleoyloxy-2-(6-carboxyspermyl)-propyl-amid (DOSPER); tetramethyltetrapalmitoyl spermine (TMTPS); (tetramethyltetraoleyl spermine (TMTOS); tetramethlytetralauryl spermine (TMTLS); tetramethyltetramyristyl spermine (TMTMS); tetramethyldioleyl spermine (TMDOS); diphytanoylphosphatidyl-ethanolamine (DPhPE); and (±)-N-(3-aminopropyl)-N,N-dimethyl-2,3-bis(dodecyloxy)-1-propanaminium bromide (GAP-DLRIE).

In other embodiments, the modulator is selected from the group consisting of: chitosan; a chitosan derivative; 1,5-dimethyl-1,5-diazaundecamethylene polymethobromide; polyoxyethylene/polyoxypropylene block copolymers; poly-L-lysine; polyamidoamine (PAMAM); PEG-block-PLL-dendrimers; polyethylenimine (PEI); mannose-PEI; transferin-PEI; linera-PEI (IPEI); gelatin; methacrylate/methacrylamide; poly(beta-amino esters); polyelectrolyte complexes (PEC); poly(vinalyamine) (PVA); Collagen; polypropylene imine (PPI); polyallylamine; polyvinylpyridine; aminoacetalized polyvinyl alcohol); acrylic or methacrylic polymer; Newkome dendrimer; polyphenylene; dimethyldioctadecylammonium bromide (DAB); cetyltrimethylammonium bromide (CTAB); albumin; acid-treated gelatin; polylysine; polyornithine; polyarginine; DEAE-cellulose; DEAE-dextran; and poly(N,N-dimethylaminoethylmethacrylate; and polypropylamine (POPAM).

In one embodiment, the modulator is selected from chitosan and chitasan derivatives. Chitosan or water soluble; chitosan nanoparticles derived from these polymers (such as described in U.S. Pat. No. 6,475,995; US 2006/0013885; Limpeanchob et al, (2006) Efficacy and Toxicity of Amphotericin B-Chitosan Nanoparticles; Nareusan University Journal 14(2):27-34). In certain embodiments, the primary amines on the chitosan polymer can be substantially modified to alter the water solubility and charge state. Chitosan derivatives include trimethyl chitosan chloride (TMC), which can be synthesized at different degrees of quaternization; mono-carboxymethylated chitosan (MCC) which is a polyampholytic polymer; glutaraldehyde cross-linked derivative (CSGA); thiolated chitosan (Lee, et al. (2007) Pharm. Res. 24:157-67); glycol chitosan (GC), a chitosan derivative conjugated with ethylene glycol (Lee, et al. (2007) Int J. Pharm.); [N-(2-carboxybenzyl)chitosan (CBCS) (Lin, et al. (2007) Carbohydr Res. 342(1):87-95); a beta-cyclodextrin-chitosan polymer (Venter, et al. (2006) Int J. Pharm. 313(1-2):36-42); O-carboxymethyl chitosan; N,O-carboxymethylchitosan; or a chitosan chemically modified by introducing xanthate group onto its backbone.

In certain embodiments, the invention provides a composition comprising a modulator as described herein. The composition can also comprise a pharmaceutically acceptable carrier. In certain embodiments, the composition does not include any nucleic acids.

Standard binding assays can be used to identify and select modulators of the invention. Nonlimiting examples are gel shift assays and BIACORE assays. That is, test modulators can be contacted with the aptamers to be targeted under test conditions or typical physiological conditions and a determination made as to whether the test modulator in fact binds the aptamer. Test modulators that are found to bind the aptamer can then be analyzed in an appropriate bioassay (which will vary depending on the aptamer and its target molecule, for example coagulation tests) to determine if the test modulator can affect the biological effect caused by the aptamer on its target molecule.

The Gel-Shift assay is a technique used to assess binding capability. For example, a DNA fragment containing the test sequence is first incubated with the test protein or a mixture containing putative binding proteins, and then separated on a gel by electrophoresis. If the DNA fragment is bound by protein, it will be larger in size and its migration will therefore be retarded relative to that of the free fragment. For example, one method for a electrophoretic gel mobility shift assay can be (a) contacting in a mixture a nucleic acid binding protein with a non-radioactive or radioactive labeled nucleic acid molecule comprising a molecular probe under suitable conditions to promote specific binding interactions between the protein and the probe in forming a complex, wherein said probe is selected from the group consisting of dsDNA, ssDNA, and RNA; (b) electrophoresing the mixture; (c) transferring, using positive pressure blot transfer or capillary transfer, the complex to a membrane, wherein the membrane is positively charged nylon; and (d) detecting the complex bound to the membrane by detecting the non-radioactive or radioactive label in the complex.

The Biacore technology measures binding events on the sensor chip surface, so that the interactant attached to the surface determines the specificity of the analysis. Testing the specificity of an interaction involves simply analyzing whether different molecules can bind to the immobilized interactant. Binding gives an immediate change in the surface plasmon resonance (SPR) signal, so that it is directly apparent, whether an interaction takes place or not. SPR-based biosensors monitor interactions by measuring the mass concentration of biomolecules close to a surface. The surface is made specific by attaching one of the interacting partners. Sample containing the other partner(s) flows over the surface: when molecules from the sample bind to the interactant attached to the surface, the local concentration changes and an SPR response is measured. The response is directly proportional to the mass of molecules that bind to the surface.

SPR arises when light is reflected under certain conditions from a conducting film at the interface between two media of different refractive index. In the Biacore technology, the media are the sample and the glass of the sensor chip, and the conducting film is a thin layer of gold on the chip surface. SPR causes a reduction in the intensity of reflected light at a specific angle of reflection. This angle varies with the refractive index close to the surface on the side opposite from the reflected light. When molecules in the sample bind to the sensor surface, the concentration and therefore the refractive index at the surface changes and an SPR response is detected. Plotting the response against time during the course of an interaction provides a quantitative measure of the progress of the interaction. The Biacore technology measures the angle of minimum reflected light intensity. The light is not absorbed by the sample: instead the light energy is dissipated through SPR in the gold film. SPR response values are expressed in resonance units (RU). One RU represents a change of 0.0001° in the angle of the intensity minimum. For most proteins, this is roughly equivalent to a change in concentration of about 1 pg/mm2 on the sensor surface. The exact conversion factor between RU and surface concentration depends on properties of the sensor surface and the nature of the molecule responsible for the concentration change.

There are a number of other assays that can determine whether an oligonucleotide or analogue thereof, peptide, polypeptide, polymer, oligosaccharide, small molecule, lipid, nanoparticle or microsphere can bind to the aptamer in a manner such that the interaction with the target is modified. For example, electrophoretic mobility shift assays (EMSAs), titration calorimetry, scintillation proximity assays, sedimentation equilibrium assays using analytical ultracentrifugation (see for eg. www.cores.utah.edu/interaction), fluorescence polarization assays, fluorescence anisotropy assays, fluorescence intensity assays, fluorescence resonance energy transfer (FRET) assays, nitrocellulose filter binding assays, ELISAs, ELONAs (see, for example, U.S. Pat. No. 5,789,163), RIAs, or equilibrium dialysis assays can be used to evaluate the ability of an agent to bind to an aptamer. Direct assays in which the interaction between the agent and the aptamer is directly determined can be performed, or competition or displacement assays in which the ability of the agent to displace the aptamer from its target can be performed (for example, see Green, Bell and Janjic, Biotechniques 30(5), 2001, p 1094 and U.S. Pat. No. 6,306,598). Once a candidate modulating agent is identified, its ability to modulate the activity of an aptamer for its target can be confirmed in a bioassay. Alternatively, if an agent is identified that can modulate the interaction of an aptamer with its target, such binding assays can be used to verify that the agent is interacting directly with the aptamer and can measure the affinity of said interaction.

In another embodiment, mass spectrometry can be used for the identification of a modulator that binds to an aptamer, the site(s) of interaction between the modular and the aptamer, and the relative binding affinity of agents for the aptamer (see for example U.S. Pat. No. 6,329,146, Crooke et al). Such mass spectral methods can also be used for screening chemical mixtures or libraries, especially combinatorial libraries, for individual compounds that bind to a selected target aptamer that can be used as modulators of the aptamer. Furthermore, mass spectral techniques can be used to screen multiple target aptamers simultaneously against, e.g. a combinatorial library of compounds. Moreover, mass spectral techniques can be used to identify interaction between a plurality of molecular species, especially “small” molecules and a molecular interaction site on a target aptamer.

In vivo or in vitro assays that evaluate the effectiveness of a modulator in modifying the interaction between an aptamer and a target are specific for the disorder being treated. There are ample standard assays for biological properties that are well known and can be used. Examples of biological assays are provided in the patents cited in this application that describe certain aptamers for specific applications and such patents are incorporated herein by reference.

The present invention also provides methods to identify the modulators of aptamers. Modulators can be identified in general, through binding assays, molecular modeling, or in vivo or in vitro assays that measure the modification of biological function. In one embodiment, the binding of a modulator to an aptamer is determined by a gel shift assay. In another embodiment, the binding of a modulator to an aptamer is determined by a Biacore assay.

In one embodiment, the modulator has the ability to substantially bind to an aptamer in solution at modulator concentrations of less than one (1.0) micromolar (uM), preferably less than 0.1 uM, and more preferably less than 0.01 uM. By “substantially” is meant that at least a 50 percent reduction in target biological activity is observed by modulation in the presence of the target, and at 50% reduction is referred to herein as an IC₅₀ value.

### Pharmaceutical Compositions

The aptamers or modulators of the invention can be formulated into pharmaceutical compositions that can include a pharmaceutically acceptable carrier, diluent or excipient. The precise nature of the composition will depend, at least in part, on the nature of the aptamer and/or modulator, including any stabilizing modifications, and the route of administration. Generally, the aptamer or modulator is administered subcutaneously or intravenously as appropriate.

In certain embodiments, the aptamer is administered in a composition that is not designed for extended release. In certain embodiments, the aptamer is administered as a pegylated form in a composition that is not designed for extended release. In certain embodiments, the aptamer is not administered in a depot, a polymeric carrier, a capsule or microcapsule, or other carrier that provides extended release when injected intravenously or intramuscularly.

Pharmaceutically useful compositions comprising an aptamer or modulator of the present invention can be formulated according to known methods such as by the admixture of a pharmaceutically acceptable carrier. Examples of such carriers and methods of formulation can be found in Remington's Pharmaceutical Sciences. To form a pharmaceutically acceptable composition suitable for effective administration, such compositions will contain an effective amount of the aptamer or modulator. Such compositions can contain admixtures of more than one compound.

In the methods of the present invention, the compounds can form the active ingredient, and are typically administered in admixture with suitable pharmaceutical diluents, excipients or carriers (collectively referred to herein as “carrier” materials) suitably selected with respect to the intended form of administration, that is, oral tablets, capsules, elixirs, syrup, suppositories, gels and the like, and consistent with conventional pharmaceutical practices.

For parenteral administration, sterile suspensions and solutions are desired. Isotonic preparations that generally contain suitable preservatives are employed when intravenous administration is desired.

The compounds of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of the present invention can also be coupled with soluble polymers as targetable drug carriers. Such polymers can include polyvinyl-pyrrolidone, pyran copolymer, polyhydroxypropylmethacryl-amidephenol, polyhydroxy-ethylaspartamidepbenol, or polyethyl-eneoxidepolylysine substituted with palmitoyl residues. Furthermore, the compounds of the present invention can be coupled (preferably via a covalent linkage) to a class of biodegradable polymers useful in achieving controlled release of a drug, for example, polyethylene glycol (PEG), polylactic acid, polyepsilon caprolactone, polyhydroxy butyric acid, polyorthoesters, polyacetals, polydihydro-pyrans, polycyanoacrylates and cross-linked or amphipathic block copolymers of hydrogels. Cholesterol and similar molecules can be linked to the aptamers to increase and prolong bioavailability.

The compounds can be administered directly (e.g., alone or in a liposomal formulation or complexed to a carrier (e.g., PEG)) (see for example, U.S. Pat. No. 6,147,204, U.S. Pat. No. 6,011,020). In one embodiment, a plurality of modulators can be associated with a single PEG molecule. The modulator can be to the same or different aptamer. In embodiments where there are multiple modulators to the same aptamer, there is an increase in avidity due to multiple binding interactions with the aptamer. In yet a further embodiment, a plurality of PEG molecules can be attached to each other. In this embodiment, one or more modulators to the same aptamer or different aptamers can be associated with each PEG molecule. This also results in an increase in avidity of each modulator to its target.

Lipophilic compounds and non-immunogenic high molecular weight compounds with which the modulators of the invention can be formulated for use in the present invention and can be prepared by any of the various techniques presently known in the art or subsequently developed. Typically, they are prepared from a phospholipid, for example, distearoyl phosphatidylcholine, and may include other materials such as neutral lipids, for example, cholesterol, and also surface modifiers such as positively charged (e.g., sterylamine or aminomannose or aminomannitol derivatives of cholesterol) or negatively charged (e.g., diacetyl phosphate, phosphatidyl glycerol) compounds. Multilamellar liposomes can be formed by the conventional technique, that is, by depositing a selected lipid on the inside wall of a suitable container or vessel by dissolving the lipid in an appropriate solvent, and then evaporating the solvent to leave a thin film on the inside of the vessel or by spray drying. An aqueous phase is then added to the vessel with a swirling or vortexing motion which results in the formation of MLVs. UVs can then be formed by homogenization, sonication or extrusion (through filters) of MLV's. In addition, UVs can be formed by detergent removal techniques. In certain embodiments of this invention, the complex comprises a liposome with a targeting aptamer(s) associated with the surface of the liposome and an encapsulated therapeutic or diagnostic agent. Preformed liposomes can be modified to associate with the aptamers. For example, a cationic liposome associates through electrostatic interactions with the nucleic acid. Alternatively, a nucleic acid attached to a lipophilic compound, such as cholesterol, can be added to preformed liposomes whereby the cholesterol becomes associated with the liposomal membrane. Alternatively, the nucleic acid can be associated with the liposome during the formulation of the liposome.

### Methods of Administration

Preferred modes of administration of the materials of the present invention to a mammalian host are subcutaneous administration of the anti-coagulant aptamer and subcutaneous or intravenous administration of the modulator. In one embodiment, the agent and carrier are administered in a slow release formulation such as an implant, bolus, microparticle, microsphere, nanoparticle or nanosphere. For standard information on pharmaceutical formulations, see Ansel, et al., Pharmaceutical Dosage Forms and Drug Delivery Systems, Sixth Edition, Williams & Wilkins (1995). Generally, pharmaceutically useful compositions useful for subcutaneous injection comprising the aptamer of the present invention can be formulated according to known methods such as by the admixture of a pharmaceutically acceptable carrier. Examples of such carriers and methods of formulation can be found in Remington's Pharmaceutical Sciences. In some embodiments, the compositions can contain admixtures of more than one aptamer.

Advantageously, aptamers can be administered in a single dose, preferably less than every day. Thereafter, the modulator is provided by any suitable means to alter the effect of the aptamer. The less than daily subcutaneous administration of the aptamer can provide a sustained level of anti-coagulant activity that can be temporarily or permanently reversed by administration of an appropriate amount of modulator.

The therapeutic compositions comprising polypeptide modulators of the present invention are conventionally administered intravenously, as by injection of a unit dose for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent; i.e., carrier or vehicle.

The compositions are administered in a manner compatible with the dosage formulation, and in a therapeutically effective amount as described herein. Suitable regimes for administration are variable, but are typified by an initial administration followed by repeated doses at less than daily intervals by a subsequent injection. In the case of modulators, the administration is intermittent or on an ‘as needed’ basis.

As used herein, the terms “pharmaceutically acceptable,” “physiologically tolerable,” and grammatical variations, thereof, as they refer to compositions, carriers, diluents and reagents, are used interchangeably and represent that the materials are capable of administration without substantial or debilitating toxic side effects.

Pharmaceutically useful compositions, comprising a modulator of the present invention can be formulated according to known methods such as; by the admixture of a pharmaceutically acceptable carrier. Examples of such carriers and methods of formulation can be found in Remington's Pharmaceutical Sciences. To form a pharmaceutically acceptable composition suitable for effective administration, such compositions will contain an effective amount of the modulator. Such compositions can contain admixtures of more than one modulator.

### Thrombotic Disorders

In some embodiments, the aptamer is administered to a patient at risk of or suffering from a thrombotic disorder. Thrombotic disorders include venous thrombosis, a condition in which a blood clot forms in a vein, which can limit blood flow and cause swelling and pain. Most commonly, venous thrombosis occurs in the “deep veins” in the legs, thighs, or pelvis (deep vein thrombosis, or DVT). Venous thrombosis can lead to a pulmonary embolism; therefore, in one embodiment, the aptamers are administered to reduce the risk of a pulmonary embolism in a patient in need thereof. Venous thrombosis can also lead to transient ischemic attacks or premature stroke; therefore, in one embodiment, the aptamers are administered to reduce the risk of ischemic attacks or strokes in a patient in need thereof.

In some embodiments, the host (or patients) are suffering from a peripheral vascular disease, particularly lower extremity occlusive disease are at higher risk; however, certain conditions including pregnancy also increase the risk of venous thrombosis. In some embodiments, the patients have a known genetic cause of a thrombotic disorder. These include a Factor V mutation (APC resistance), Protein S deficiency, Protein C deficiency, Antithrombin III deficiency, Plasminogen deficiency or Heparin cofactor II deficiency. In other embodiments, the patient is at risk of a thrombotic disorder due to an acquired or environmental conditions that can precipitate a thrombotic event, including pregnancy, oral contraceptive use, estrogen therapy, obesity, a malignancy, diabetes mellitus, venous stasis from immobility, trauma, a post-operative state, and Lupus anticoagulant.

### EXAMPLES

### Materials and Methods

### Measures of Testing Coagulation

Standard measures of coagulation include the plasma-based prothrombin time (PT) assays, plasma and whole blood-based activated partial thromboplastin time (APTT) assays, and the whole blood-based activated clotting time (ACT) assay. While the activators used to initiate coagulation in each of these assays are different, they share the common feature of clot formation as the endpoint for the assay. Importantly, in these in vitro, assays, low levels of thrombin, ˜10-30 nM, are sufficient to: produce enough fibrin to reach the endpoint. This level of thrombin represents conversion of only 3-5% of prothrombin to thrombin, and is consistent with the amount of thrombin generated during the initiation phase of the coagulation reaction (Butenas et al., (2003) J Thromb Haemost. 1:2103-11; Mann et al., (2003) Arterioscler Thromb Vase Biol. 23:17-25.). Thus, these assays report largely on the initiation phase of the coagulation reaction, and do not fully reflect the impact of a deficiency in, or inhibition of, coagulation factors primarily involved in the propagation phase of coagulation.

The manner in which the standard clot-based assays reflect FIX/IXa activity is exemplified by their ability to detect or not detect abnormal, coagulation measures in individuals with severe hemophilia A (a FVIII deficiency) or B (a FIX deficiency). A hallmark of hemophilia is the isolated prolongation of the APTT, as individuals with hemophilia have abnormal APTTs, but normal PTs (Bolton-Maggs and Pasi, (2003) Lancet. 361:1801-9). The cell-based model of coagulation explains the paradox as to why individuals deficient in FVIII or FIX register normal PTs. The PT assay is initiated with supra-physiologic levels of tissue factor, enough to yield a clot in 11-15 seconds. Therefore, the high levels of tissue factor-FVIIa complex used to initiate the reaction rapidly produce FXa in amounts sufficient to yield enough thrombin to reach the clot endpoint, even in the absence of FVIII or FIX. Thus, even profound inhibition of FIX/FIXa activity is not expected to impact a PT assay, as the role of FIX in the initiation of coagulation is masked, or bypassed, in this assay. Thus, pharmacologic inhibitors of FIXa, such as the anti-FIXa aptamer RB006, are not expected to prolong PT values.

Both plasma and whole blood APTT assays are initiated with a charged particulate, such as celite or kaolin, a phospholipid surface, and calcium in sufficient quantities to yield a clot in ˜28-35 seconds. Individuals with hemophilia B (and A) register abnormal APTT values; however, the magnitude of the prolongation of APTT in these individuals is finite (i.e., yields a limited value), as the assay largely reports on the initiation phase of coagulation. There is not a tight correlation between the severity of an individual's hemophilia B and their A PIT value, as the APTT is dependent upon other coagulation factors in addition to FIX. Therefore, a better framework for interpreting how pharmacologic inhibition of FIXa is expected to register in the APTT assay is the plasma FIX assay. The plasma FIX assay is a variation of the standard APTT method in which test plasma is diluted in buffer and mixed with FIX-deficient plasma prior to performing the APTT, such that the FIX level in the test plasma is the primary determinant of the clot time. This assay is typically used to determine the severity of hemophilia B (i.e., determine FIX levels) or to diagnose acquired inhibitors of FIX. The results of the FIX assay arc interpreted by comparing the clot time of the lest sample to a FIX-level standard curve, which is prepared by serial dilution of normal plasma in buffer prior to mixing, with FIX-deficient plasma. Table 1 shows a typical FIX level standard curve performed with normal human plasma. As observed in Table 1, at levels of FIX that are 25% normal (i.e., reduced 75%), APTT clot times are increased 1.4-fold above baseline. At FIX levels ˜3% normal (i.e., reduced by 97%), APTT clot times are increased 2-fold above baseline, and at FIX levels <1% normal (i.e., reduced >99%), APTT clot times are increased 2.5 fold relative to baseline. Carriers of hemophilia B (i.e. ˜50% normal FIX levels) exhibit normal APTT values, which is consistent with the data from the FIX level standard curve. Taken together, these observations indicate that a significant percentage of FIX activity must be inhibited before the APTT will be prolonged.

TABLE 1
FIX Activity Assay Standard Curve in Human Plasma

| % FIX Level | APTT Clot Time | Fold increase in Clot Time |
|---|---|---|
| 100* | 48.0 | 1.0 |
| 50 | 58.6 | 1.2 |
| 25 | 65.4 | 1.4 |
| 12.5 | 75.1 | 1.6 |
| 6.25 | 85.1 | 1.8 |
| 3.13 | 97.0 | 2.0 |
| 1.56 | 105.8 | 2.2 |
| 0.78 | 119.7 | 2.5 |

*100% FIX level represents a 1:5 dilution of normal pooled human plasma in buffer

The ACT is a clotting endpoint assay initiated, with charged particulates, thus the effect of pharmacologic inhibition of FIXa in the ACT assay likely mirrors that observed in the APTT assay. That is, it is anticipated that prolongation of the ACT will not be observed until a substantial degree of FIXa inhibition is reached (>50%). Hence, analogous to the APTT assay, the magnitude of the prolongation of the ACT is likely to be modest as compared to the prolongation observed with unfractionated heparin. Finally, the assay is likely to saturate in response to FIXa inhibition.

A 3-part study was undertaken 1) to assess the adsorption kinetics and pharmacodynamic effects of RB006 following subcutaneous administration; 2) the ability of the modulator RB007 administered intravenously to reverse the effects of RB006 administered subcutaneously; and 3) to assess the repeat-dose tolerability of RB006 administered subcutaneously over a 7 day period. A total of four monkeys were assigned to this study, and were rotated through the 3 phases of the study with appropriate wash-out periods between treatments.

### Example 1

### Exploratory Time Course of RB006 in Cynomolgus Monkeys

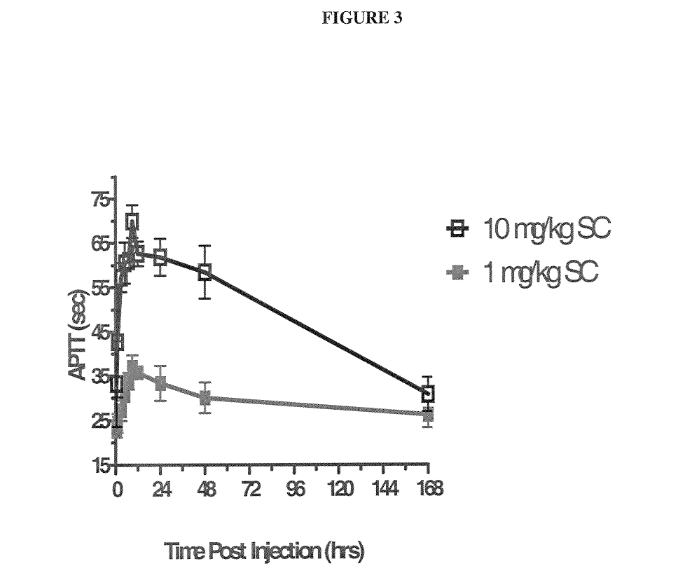

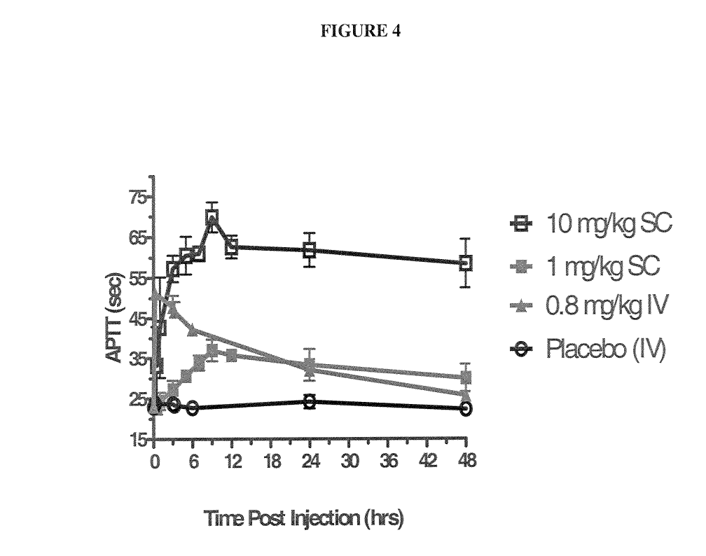

Previous studies have shown that one can expect a steady decrease in plasma level concentrations following intravenous or subcutaneous administration of certain aptamers (Tucker et al. Journal of Chromatography B, 732:203-212, 1999). The time course of RB006 concentration was evaluated following subcutaneous injection in Cynomolgus monkeys. Four monkeys were given a single subcutaneous injection of RB006 at two different dose levels (2 monkeys/group). Group 1 received 1 mg/kg of RB006 subcutaneously. Group 2 received 10 mg/kg of RB006 administered subcutaneously. Serial blood samples were collected and assessed for anti-coagulant activity and plasma concentration using the concentration-dependent prolongation of time-to-clot APTT assay. As shown in FIG. 3, animals assigned to both Group 1 and Group 2 demonstrated sustained anti-coagulant activity over the initial 48 hours before falling off to base line levels within one week. For comparison, FIG. 4 contains data from a prior study in which monkeys received RB006 at 0.8 mg/kg or placebo administered intravenously. Monkeys that received intravenously administered RB006 exhibited a rapid onset of anti-coagulant activity that also rapidly decreased with a return to base line levels within 48 hours. This is in contrast to RB006 administered subcutaneously in which the onset of APTT prolongation was delayed and gradual (for a few hours at the 1 mg/kg dose although somewhat more rapid at the 10 mg/kg dose). Prolongation was maximal at 9 to 12 hours post injection at the 1 mg/kg dose and was still substantially prolonged by 48 hours post-dose in the 10 mg/kg dose, which apparently reflected both a protracted absorption phase and a long circulation half-life for RB006. Measurement of the plasma concentration of RB006 supports the observed pharmacodynamic effects (see Table 2).

TABLE 2
Mean Plasma Concentrations of RB006 in Cynomolgus Monkeys (μg/mL)
Example 1 - Subcutaneous injection of RB006

| Group | RB006 Dose | Time Post RB006 Dose (hours) | | | | | | | | |
|---|---|---|---|---|---|---|---|---|---|---|
| No. | (mg/kg) | 0.5 | 1 | 3 | 5 | 7 | 9 | 12 | 24 | 48 |
| 1 | 1 | 0.35 | 1.21 | 4.17 | 5.62 | 8.57 | 9.59 | 10.89 | 12.37 | 9.74 |
| 2 | 10 | 5.95 | 14.66 | 32.22 | 54.37 | 62.07 | 91.07 | 90.04 | 103.2 | 72.12 |

The pre-study samples that were collected prior to Example 1 contained no RB006 (data not shown). Following subcutaneous injection of 1 or 10 mg/kg RB006 as discussed in Example 1 (Groups 1 and 2, respectively), RB006 was detected in plasma at the first sampling time point (0.5 hours), and the levels increased gradually over time, reaching a maximum al 24 hours post-dosing for both dose levels. However, the mean concentrations at 9 hours post-dose were 78 and 88 percent of the maximal (24-hour) levels for the 1 and 10 mg/kg doses, respectively. By 48 hours post-dose, mean RB006 levels had decreased only slightly from the 24-hour levels. Hence, the systemic absorption of RB006 following subcutaneous injection is quite long, which, in combination with an approximate 12-hour half-life in circulation (as documented in a previous study; data not shown), results in accumulation in plasma over a 24-hour period following injection and persistence through 48 hours post-dose. Across time, the plasma concentrations of RB006 were approximately dose-proportional between the 1 and 10 mg/kg doses. These results indicate substantially increased RB006 stability and/or plasma residence times following subcutaneous administration as compared to intravenous administration.

### Example 2

### Assessment of Modulator Efficacy

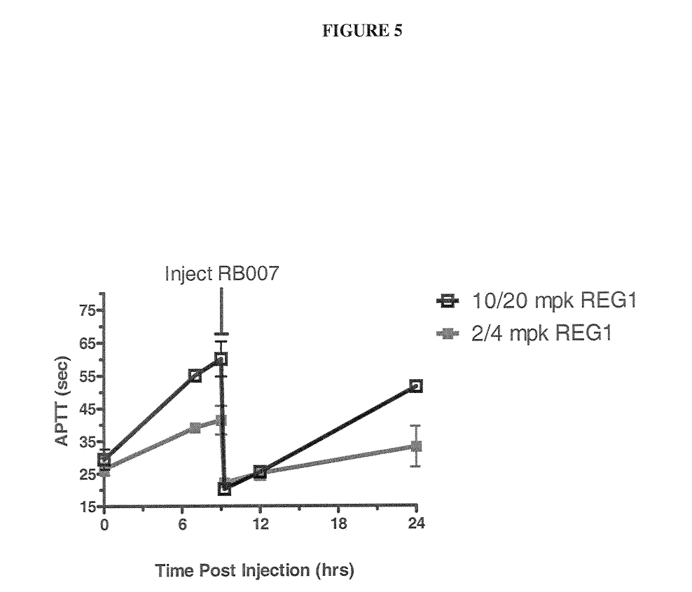

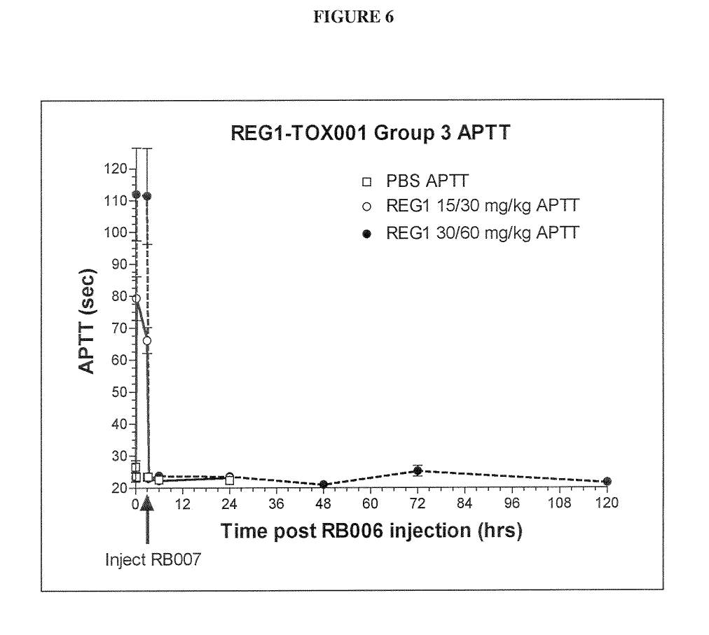

Following a 7 day washout period, the same animals from Example 1 received a single subcutaneous dose of RB006. Group 1 received 2 mg/kg and Group 2 received 10 mg/kg. Nine hours post-injection, the modulator RB007 was injected as a single bolus intravenously. Group 1 received 4 mg/kg of RB007 and Group 2 received 20 mg/kg of RB007. As shown in FIG. 5, RB007 effectively neutralized anti-coagulant activity. However, the anti-coagulant activity returned to near pre-modulator injection levels within 16 hours. The most likely explanation for this subsequent rise in APTT following RB007 administration was that free RB007 in circulation had cleared from the blood compartment prior to complete absorption of RB006 from the subcutaneous injection site, such that APTT was again prolonged by newly absorbed RB006 after RB007 levels had declined. These data indicate that the absorption of RB006 following subcutaneous dosing may be protracted (i.e., occurring over a period greater than 12 hours post-dose) and that additional doses of RB007 may be required to permanently inactivate the RB006 taken up from the subcutaneous injection. Results from a previous toxicokinetic study as detailed in FIG. 6 had shown that administration of the RB007 modulator (30 or 60 mg/kg), subsequent to intravenous administration of RB006 (15 or 30 mg/kg), effectively neutralized aptamer activity up to 117 hours after administration of the modulator. Taken together, these unexpected findings resulting from subcutaneous injection of RB006 indicate that unlike intravenous administration, there is a slower absorption phase for RB006 as it transitions from the injection site to the plasma compartment. This may contribute to the ability of the aptamer to provide prolonged and sustained anti-coagulant activity following subcutaneous administration.

Mean RB006 plasma concentrations following the 10 mg/kg dose were very similar to what was measured in the Group 2 animals, in Example 1 that received the same dose, while the levels for the Group 1 animals that were given the 2 mg/kg-dose were approximately twice those present in the Groups 1 animals that were given 1 mg/kg, again reflecting the dose-proportional plasma pharmacokinetics for RB006 (See Table 3).

TABLE 3
Mean Plasma Concentrations of RB006 in Cynomolgus Monkeys (μg/mL)
Example 2: Subcutaneous RB006 followed by IV RB007

| | | Time Post RB006 | | | Time Post RB007 | | |
|---|---|---|---|---|---|---|---|
| Group | RB006 Dose | Dose (hours) | | | Dose (hours) | | |
| No. | (mg/kg) | predose | 7 | 9 | 0.25 | 3 | 15 |
| 1 | 2 | 3.89 | 13.87 | 22.22 | 1.08 | 3.97 | 8.31 |
| 2 | 10 | 16.54 | 67.32 | 73.44 | 0.52 | 12.57 | 42.08 |

When RB007 was administered (by intravenous injection) at 9 hours after the RB006 dose, the mean concentration of RB006 decreased dramatically by 15 minutes after the RB007 dose, to approximately 5% and 0.7% of the mean levels prior to RB007 injection for Groups 1 and 2, respectively. This rapid elimination of RB006 reflects the modulator property of RB007, which hybridizes to RB006 and neutralizes its activity. At that same time (15 minutes after RB007 injection), the plasma levels of free RB007 were very low in Group 1 animals (mean of 0.02 μg/mL) but were more substantial in Group 2 (mean of 45 μg/mL) that received the higher (20 mg/kg) dose of RB007. The higher concentration of free RB007 following the 20 mg/kg dose may explain why the free RB006 level was lower in Group 2 (vs. Group 1), even though the dose of RB006 was 5 times higher than that given to Group 1.

By 3 hours after RB007 injection, plasma levels of RB006 were increased, as compared to 15 minutes after the RB007 dose, and the RB006 levels continued to rise through 15 hours following the RB007 dose. This resurgence of the RB006 levels in plasma following modulator dosing reflects both the short plasma half-life of RB007 and the extended absorption phase for subcutaneously injected RB006 (as described in Example 1), such that new quantities of RB006 entering the circulation after the RB007 dose (given at 9 hours following the RB006 dose) are not bound by RB007, as the modulator was either consumed in binding to the RB006 present at 9 hours or was cleared rapidly thereafter. In support of this interpretation, it is noteworthy that, for Group 2, the sum of the RB006 concentration present immediately prior to RB006 dosing and that present at 15 hours after the RB007 dose (respective means of 73 and 42 μg/mL= 115 μg/mL) is similar to the maximum RB006 plasma level reached in Example 1 at the same dose level (mean of 103 μg/mL at 24 hours post-dosing), which indicates that the rise in RB006 levels at later limes after the RB007 dose is not due to reversal of the binding of RB006 to RB007, but rather to the influx of more RB006 from the subcutaneous injection site in the absence of a persistent level of RB007 in circulation. The slightly higher total amounts of RB006 in circulation in this treatment phase can be accounted for by the predose level, as the animals were apparently not completely washed out with respect to RB006 clearance (the predose means for Groups 1 and 2 prior to this experiment were 3.9 and 16.5 μg/mL, respectively).

### Example 3

### Repeat-Dose Tolerability Study

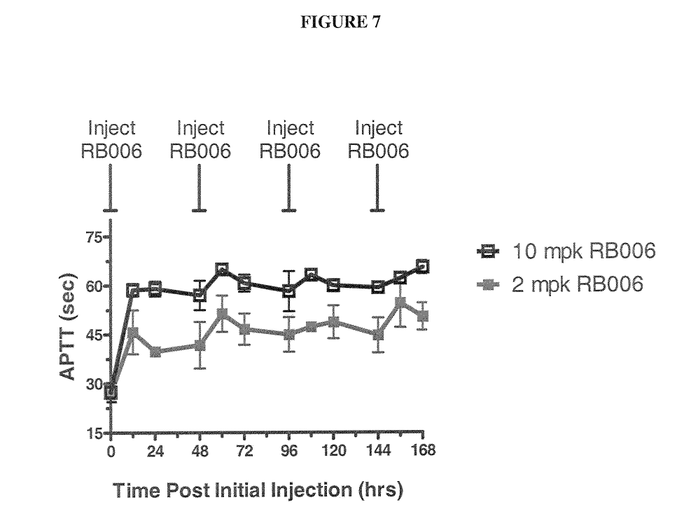

Following an additional 7 day washout period, injection site tolerability and time course activity following multiple repeat dosing was assessed. Groups 1 and 2 received repeated (4 total) single subcutaneous injections of RB006 at 2 and 10 mg/kg respectively. Serial blood samples were collected and assessed for anti-coagulant activity and plasma concentration using the concentration-dependent prolongation of time-to-clot APTT assay. As show in FIG. 7, at both dose levels it was possible to maintain a steady state of anticoagulant activity for a week using subcutaneous injection of RB006 every 48 hours. With this repeated RB006 dosing, APTT was markedly prolonged throughout the dosing period. The prolongation of APTT was dose-dependent, such that the animals treated with 10 mg/kg/dose had APTT values approximately 25-30% higher than the values for the animals treated with 2 mg/kg/dose (Group. 1). The APTT prolongation-observed in those animals receiving 10 mg/kg/dose was at the high end of prolongation that can be produced by RB006, reflecting complete inhibition of Factor IX in the monkeys, while the APTT prolongation in the 2 mg/kg/dose was close to the maximum effect of RB006.

When RB006 was given as 4 repeated subcutaneous injections every other day for 4 doses, the plasma pharmacokinetics were consistent with those obtained in Examples 1 and 2 (see Table 4).

Table 4: Mean Plasma Concentrations of RB006 in Cynomolgus Monkeys (μg/mL)

Example 3: Repeated Subcutaneous RB006

| | | Time Post RB006 Dose (hours) | | | | | | |
|---|---|---|---|---|---|---|---|---|
| Group | RB006 Dose | 1st Dose | | | | 2nd Dose | | |
| No. | (mg/kg) | predose | 12 | 24 | 48 | 12 | 24 | 48 |
| 1 | 2 | 3.28 | 21.04 | 29.45 | 24.64 | 39.73 | 43.95 | 33.91 |
| 2 | 10 | 8.03 | 72.87 | 103.11 | 90.54 | 352.34 | 369.43 | 230.98 |

| | | | |
|---|---|---|---|
| Group | RB006 Dose | 3rd Dose | Last Dose |

| No. | (mg/kg) | 12 | 24 | 48 | 12 | 24 |
|---|---|---|---|---|---|---|
| 1 | 2 | 45.16 | 43.18 | 67.65 | 58.20 | 67.02 |
| 2 | 10 | 122.32 | 308.32 | 124.90 | 210.79 | 191.57 |

At 12 hours after the first dose of RB006, mean plasma levels were higher than the levels measured in the animals in Examples 1 and 2 at the same dose levels, but this was attributable to incomplete washout of RB006 prior to initiating this study (the predose means for Groups 1 and 2 were 3.3 and 8.0 μg/mL. respectively). RB006 continued to accumulate in plasma through 24 hours after the first dose, and then diminished slight at 48 hours, as seen in Example 1. Consequently, there was substantial accumulation of RB006 in plasma with every-other-day dosing, particularly between the first and second doses. However, the peak plasma levels following the third dose of 10 mg/kg (at 24 hours post-dose) were actually slightly lower than after the second dose, and they were lower still after the last dose (but higher than after the first dose). This time course suggests that, for the higher dose level of 10 mg/kg dose, RB006 levels in plasma had reached steady state by the third dose and/or that there was an increase in the clearance rate of RB006 with repeated dosing. For the lower dose level of 2 mg/kg/dose, accumulation was evident through the third dose (with a peak at 48 hours following this dose) and there was a slight additional accumulation (when comparing levels at the same time points) between the third and fourth doses. This difference in repeat-dose kinetics between the two dose levels may reflect dose-dependence differences in RB006 accumulation, but the means were based on only two animals.

These data indicate it is possible to sustain anti-coagulant activity in a host using less than daily doses of RB006 subcutaneously. This unexpected finding has direct implications on the ability to treat chronic thrombotic disorders like venous thrombosis, which require prolonged and sustainable levels of anti-coagulant activity. In addition, the ability to administer the aptamer effectively through less than daily subcutaneous injections offers greater flexibility in administering and treating thrombotic disorders than previously allowed by intravenous methods.

1. A method of enhancing or increasing plasma half life of an aptamer comprising administering the aptamer subcutaneously to a host.

2. The method of claim 1 further comprising administering a modulator agent.

3. The method of claim 1, wherein the method provides reduction or prevention of thrombosis in a host in need thereof.

4. The method of claim 1 wherein the aptamer regulates a factor in the coagulation cascade.

5. The method of claim 1 wherein the aptamer regulates a coagulation factor IX or IXa activity in the host.

6. The method of claim 3 wherein the aptamer is administered in a single subcutaneous dose less than once a day.

7. The method of claim 1 wherein the aptamer comprises RB006 (SEQ ID NO:1).

8. The method of claim 7 wherein the aptamer further comprises at least one polyethylene glycol molecule.

9. The method of claim 2 wherein the modulator reverses an activity of the aptamer in the host.

10. The method of claim 2 wherein the modulator partially reverses an activity of the aptamer in the host.

11. The method of claim 9 wherein the activity is anticoagulant activity.

12. The method of claim 9 wherein the activity is inhibition of coagulation factor IXa.

13. The method of claim 9 wherein the modulator is an oligonucleotide.

14. The method of claim 10 wherein the activity is anticoagulant activity.

15. The method of claim 10 wherein the activity is inhibition of coagulation factor IXa.

16. The method of claim 10 wherein the modulator is an oligonucleotide.

17. The method of claim 2 wherein the modulator is administered intravenously.

18. The method of claim 2 wherein the modulator comprises RB007 (SEQ ID NO:2).

19. The method of claim 2 wherein the aptamer is administered in a subcutaneous depot for extended release.

20. A method for administering an effective dose of an anticoagulant aptamer comprising: 1) determining a dose for a desired pharmacodynamic response; and 2) administrating to a host in need thereof a dose of aptamer anticoagulant to achieve the desired pharmacodynamic response, where the aptamer is administered in a single subcutaneous dose less than once a day.

21. The method of claim 20 wherein the aptamer comprises RB006 (SEQ ID NO:1).

22. The method of claim 20 further comprising administering a modulator to the host.

23. The method of claim 22 wherein the modulator comprises RB007 (SEQ ID NO:2).
